(19) United States
(12) Patent Application Publication
(10) Pub. No.: US 2021/0219947 A1
(43) Pub. Date: Jul. 22, 2021

(54) Intraoperative Ultrasound Probe System and Related Methods

(71) Applicant: Tissue Differentiation Intelligence, LLC, Delray Beach, FL (US)

(72) Inventor: Adam Azzara, San Diego, CA (US)

(21) Appl. No.: 17/151,648

(22) Filed: Jan. 18, 2021

Related U.S. Application Data
(60) Provisional application No. 62/962,160, filed on Jan. 16, 2020.

(51) Int. Cl. A61B 8/08; A61B 8/00

(52) U.S. Cl. CPC A61B 8/0841; A61B 8/483; A61B 8/4488; A61B 8/4218; A61B 8/5261; A61B 8/463; A61B 8/469; A61B 8/4263; A61B 8/0875; A61B 8/54

(57) ABSTRACT

An intraoperative ultrasound imaging system and method capable of using ultrasound imaging to safely place a surgical access instrument (e.g. guide wire, dilator, cannula, etc.) through a tissue (e.g., muscle, fat, brain, liver, lung, etc.) without damaging nearby neurovascular structure is described herein. The intraoperative ultrasound system includes an ultrasound probe assembly configured for emitting and receiving ultrasound waves and a computer system including a processor and a display unit. Once the probe is in position, ultrasound imaging is performed wherein the computer receives RF data from the probe and causes a B-mode image of the visible anatomical structures (e.g. muscle, bone, etc.) to be displayed on the display unit.

### CROSS-REFERENCES TO RELATED APPLICATIONS

The present application is a nonprovisional application claiming benefit of priority from commonly owned and co-pending U.S. Provisional Application Serial No. 62/962,160 filed on Jan. 16, 2020 and entitled “INTRAOPERATIVE ULTRASOUND PROBE SYSTEM AND RELATED METHODS,” the entire contents of which is hereby incorporated by reference into this disclosure as if set forth fully herein.

### FIELD

The present disclosure relates generally to ultrasound imaging, and more specifically to a system and method for using ultrasound imaging to safely place one or more instruments through tissue without damaging nearby neurovascular structures.

### BACKGROUND

Ultrasound imaging or sonography is a diagnostic medical procedure that uses high-frequency sound waves to produce visual images of anatomical features inside the body, including but not limited to organs, tissue, and blood flow. As a result, ultrasound imaging can be a useful tool for providing a real-time visualization of internal anatomy prior to accessing a surgical target site. However, traditional ultrasound techniques are not effective at detecting certain kinds of tissue including neural tissue, and thus traditional ultrasound techniques may be unreliable for visualization of internal anatomy for certain procedures, for example such as a lateral trans-psoas approach to the spine.

### SUMMARY

The present disclosure describes an intraoperative ultrasound probe system and method capable of using ultrasound imaging to safely guide the placement of one or more instruments (e.g., needle, guide wire, dilator, cannula, etc.) through tissue (e.g., muscle, fat, brain, liver, lung, etc.) without damaging nearby neurovascular structures. According to one embodiment, and by way of example only, the intraoperative ultrasound probe system includes an ultrasonic transducer probe configured for emitting and receiving ultrasound waves and a computer system including a processor for processing the data received by the probe and a display unit configured to display an ultrasound image based on the processed data. The ultrasonic transducer probe is configured for insertion into an operative corridor in a surgical patient so that the distal end of the ultrasonic transducer probe may be brought into close proximity or contact with target anatomy. For example, in a lateral access spine surgery the distal end of the ultrasonic transducer probe may be placed in close proximity to the psoas muscle. Ultrasound imaging may then be used to locate and identify certain anatomical structures to help the surgeon visually determine a safe trajectory for a guide wire through the psoas muscle to a target spinal disc space. Once the probe is in position proximate the psoas muscle (including but not limited to direct contact), ultrasound imaging may be performed in which the computer receives radio frequency (RF) data from the probe and causes a B-mode image of the visible anatomical structures (e.g. muscle, bone, etc.) to be displayed on the display unit.

In some embodiments, the intraoperative ultrasound probe system includes a probe configured for emitting and receiving ultrasound waves in electronic communication with an electronic device (e.g. computer) including a computer processor for processing data received by the probe, software for providing a set of executable instructions to the processor, and a display unit (integrated or standalone) configured to display an ultrasound image based on the processed data. The electronic device may be any stationary or portable computer system including a processor, software, and the ability to communicate with a display unit (integrated or standalone), including but not limited to a laptop, desktop, workstation, personal digital assistant, server, blade server, mainframe, cellular telephone, smartphone, tablet computer, cloud-based computing devices, and/or other similar computing devices. In some embodiments, the intraoperative ultrasound probe system may include include a variety of instruments and accessories, including but not limited to a transducer stabilizer, an stabilizer tube, dilator guide, dilator, and a K-wire.

In some embodiments, the ultrasonic transducer probe comprises an elongated housing member having an elongated main body portion, a distal end, a proximal end, a superior face, an inferior face, an inner cavity, and a proximal aperture through which a communication cable passes that may connect the probe to one or more of a power source, display device, computer, and the like. In some embodiments, the main body portion includes an elongated coupling track positioned on the superior face that extends substantially the length of the main body portion and may be configured to slidably couple with one or more attachments or accessories. In some embodiments, the main body portion comprises a passive locking element configured to engage one or more accessories, including but not limited to a transducer stabilizer. In some embodiments, the passive locking element comprises a recess formed within the superior face near the proximal end of the coupling track. Formation of the recess may create a pair of sidewalls positioned on either side of the recess and extending partially the length of the recess, leaving a gap between the distal ends of the sidewalls and the distal end of the recess. In some embodiments, the distal end has an enlarged width to accommodate the ultrasonic transducer array disposed within the interior cavity at the distal end of the probe. In some embodiments, the distal end further comprises a generally planar leading face having smooth rounded edges to minimize trauma to surrounding tissue as the probe is advanced and retracted through the operative corridor. In some embodiments, the proximal end includes curved portion such that the proximal end is laterally offset from the main body portion in the inferior direction.

In some embodiments, the intraoperative ultrasound probe system of the present disclosure further comprises a transducer stabilizer. In some embodiments, the distal region of the probe is sized and configured to slidingly engage the stabilizer tube. However, the proximal region, being of narrower width than the distal region (in some embodiments), does not itself engage the stabilizer tube and as a result would be free to move (and thereby change trajectory angle during use) absent a stabilizing intermediate structure. Thus the transducer stabilizer is configured to attach to the transducer probe near the proximal region and further engage the stabilizer tube such that the narrower proximal region of the probe is secured in position when the probe is inserted into the stabilizer tube.

In some embodiments, the transducer stabilizer has a main body portion comprising a superior surface, inferior surface, and a central recess formed therein that is open to one side of the stabilizer. In some embodiments, the superior surface is generally planar with smooth rounded edges such that the stabilizer has a generally rounded rectangle cross-sectional shape. In some embodiments, the central recess is sized and configured to receive a portion of the proximal region of the probe therein. In some embodiments, the stabilizer further includes a first engagement element positioned at the closed side of the recess (opposite the open side) and configured to engage the probe to couple the stabilizer to the probe, for example by engaging the passive locking element (or similar feature) on the probe. In some embodiments, the stabilizer further includes a visual indicator that will indicate a positive locking engagement between the stabilizer and probe.

In some embodiments, the transducer stabilizer may be further configured to securely engage the stabilizer tube while simultaneously coupled to the probe. To facilitate this engagement, the stabilizer may include a pair of inferior buttresses extending from the inferior surface of the main body portion. The inferior buttresses may be located at either end of the stabilizer (e.g. one on each side of the central recess) and may have a curved perimeter shape that corresponds to the perimeter shape of the interior lumen of the stabilizer tube such that the inferior buttresses are sized and shaped to be snugly received within the interior lumen of the stabilizer tube. The main body portion (including the superior and inferior surfaces) may have a slightly larger perimeter than the buttresses so as to create an overhang or lip that prevents the entire stabilizer from entering the interior lumen of the stabilizer tube. A pair of elongated flanges may extend further inferiorly into the interior lumen (when coupled to the stabilizer tube) to provide further stability. As a result, in some embodiments the transducer stabilizer may be configured to “sit” on top of the stabilizer tube when engaged thereto and maintain the proximal end of the probe in a fixed orientation relative to the stabilizer tube.

In some embodiments, the transducer stabilizer may be manufactured from a medical-grade radiolucent material such as PEEK (poly-ether-ether-ketone), PEKK (poly-ether-ketone-ketone), etc. and may further contain radiographic markers positioned to signal the location of one or more of the guide channels of the dilator guide under fluoroscopy. In some embodiments, the radiographic markers may be spot markers to indicate the location of the top openings of the guide channels of the dilator guide and/or a linear marker to indicate the alignment and/or angular orientation of the guide channels. In some embodiments, the transducer stabilizer may be made from anodized aluminum.

In some embodiments, the intraoperative ultrasound probe system of the present disclosure further comprises a stabilizer tube. In some embodiments, the stabilizer tube comprises an elongated cannulated sleeve having a proximal end, a distal end, and an interior lumen extending from the proximal end to the distal end. In some embodiments, the sleeve and the interior lumen each have a generally rounded rectangle cross-sectional shape. In some embodiments, the interior lumen may be sized and configured to receive at least a portion of the stabilizer buttresses therein at the proximal end and to further receive the dilator guide therein. In some embodiments, the sleeve further comprises a proximal aperture and a distal aperture at the respective ends of the interior lumen to enable ingress and egress of various surgical instruments through the stabilizer tube. In some embodiments, the proximal end may further include a superior flange to buttress the laterally-offset curved portion of the probe and a laterally extending flange configured to engage with an articulating arm (for example) to register the stabilizer tube (and by extension any instruments associated with it such as the probe, dilator guide, etc.) to a bedrail in a fixed orientation.

In some embodiments, the intraoperative ultrasound probe system of the present disclosure further comprises a dilator guide. In some embodiments, the dilator guide comprises an elongated cannulated sleeve having a proximal end, a distal end, and one or more guide channels in the form of interior lumens extending from the proximal end to the distal end and having proximal openings and distal openings to allow ingress and egress of various instrumentation therethrough. In some embodiments, the dilator guide may have a plurality of guide channels extending therethrough. In some embodiments, the dilator guide may have three cylindrical guide channels, including a central guide channel and a pair of lateral guide channels. In some embodiments, the guide channels may be sized and configured to receive at least one dilator therethough, however any instrument having a diameter or width smaller than the diameter of the guide channels may pass through. In some embodiments, the proximal end of the dilator guide is configured to “sit” on top of the stabilizer tube when engaged thereto and maintain the dilator guide (and importantly, the guide channels in a fixed orientation relative to the stabilizer tube. In some embodiments, the distal end of the dilator guide has a perimeter surface having a size and shape corresponding to the perimeter size and shape of the interior lumen (when coupled with the stabilizer tube) so that the distal end is snugly received within the interior lumen to provide further stability.

In some embodiments, the ultrasonic transducer probe may be cannulated to enable advancement of a surgical instrument (e.g. dilator, K-wire) through the probe. In some embodiments, the cannulation comprises an interior corridor extending substantially the length of the main body portion and configured to allow passage of one or more surgical instruments (e.g. surgical guide wire) therethrough.

In some embodiments, the surgical guide wire may include a series of echogenic elements configured to reflect sound waves to make the surgical guide wire “visible” during ultrasound imaging. In some embodiments, the echogenic elements comprise one or more of notches, ridges, and the like.

In some embodiments, the probe comprises an elongated housing member having a generally hourglass shape. In some embodiments, the elongated housing member may include an enlarged-width distal end comprising an outer surface having a curved perimeter shape that corresponds to the perimeter shape of the interior lumen of the stabilizer tube, encouraging a snug interaction between the distal end and the stabilizer tube to minimize or eliminate non-translational movement of the distal end relative to the stabilizer tube during use. In some embodiments, the elongated housing member may include an enlarged-width proximal end comprising an outer surface having a curved perimeter shape that corresponds to the perimeter shape of the interior lumen of the stabilizer tube, encouraging a snug interaction between the proximal end and the stabilizer tube to minimize or eliminate non-translational movement of the proximal end relative to the stabilizer tube during use.

In some embodiments, the probe further comprises a proximal extension comprising an inner cavity through which a communication cable passes that may connect the probe (e.g. including but not limited to the transducer array) to one or more of a power source, display device, computer, and the like. In some embodiments, the proximal extension may be curved or angled such that the proximal end is laterally offset from the distal end, creating additional space to maneuver instrumentation as the probe is advanced or retrieved from the operative corridor, for example via the stabilizer tube.

In some embodiments, the electronic device comprises a moveable unit having a computer housing (e.g. comprising a processor, software, data storage module, and a communications module configured for wired and/or wireless communication with the probe), a base unit, and a display unit coupled to a vertical displacement element. In some embodiments, the base unit has a plurality of wheel elements (e.g. castors, etc.) that enable a user to move the moveable unit to any desired position in a room. In some embodiments, the electronic device including the computer housing and display unit may be connected to a power source, either integrated with the electronic device or connected to A/C power via a power cord. In some embodiments, the electronic device maybe A/C capable with a battery backup. In some embodiments, the data storage module may include internal storage or external storage. In some embodiments, the display unit may have a screen comprising a touch-screen interface that enables the user to provide instructions to the computer by selecting buttons or icons that the computer presents on the screen.

In some embodiments, direct visualization of the dilator/K-wire placement may be utilized prior to removal of the stabilizer tube.

In some embodiments, the system may include integration of three-dimensional soft tissue mapping capabilities enabled by image-guided navigation. In some embodiments, robotic automation may be employed to enhance precision and efficiency.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a block diagram illustrating an example of an intraoperative ultrasound probe system according to one embodiment of the disclosure;

FIG. 2 is a perspective view of an example of an ultrasonic transducer probe forming part of the intraoperative ultrasound probe system of FIG. 1;

FIG. 3 is a plan view of a proximal end of the ultrasonic transducer probe of FIG. 2;

FIG. 4 is a side plan view of the ultrasonic transducer probe of FIG. 2;

FIG. 5 is a top plan view of the ultrasonic transducer probe of FIG. 2;

FIG. 6 is a bottom plan view of the ultrasonic transducer probe of FIG. 2;

FIG. 7 is a perspective view of an example of a transducer stabilizer forming part of the intraoperative ultrasound probe system of FIG. 1;

FIG. 8 is a front plan view of the transducer stabilizer of FIG. 7;

FIG. 9 is a top plan view of the transducer stabilizer of FIG. 7;

FIG. 10 is an exploded perspective view of the transducer stabilizer of FIG. 7;

FIG. 11 is a perspective view of an example of a stabilizer tube forming part of the intraoperative ultrasound probe system of FIG. 1;

FIG. 12 is a top plan view of the stabilizer tube of FIG. 11;

FIG. 13 is a side plan view of the stabilizer tube of FIG. 11;

FIG. 14 is a side sectional view of the stabilizer tube of FIG. 11, taken along line A-A in FIG. 13;

FIG. 15 is a perspective view of an example of a dilator guide forming part of the intraoperative ultrasound probe system of FIG. 1;

FIG. 16 is a top plan view of the dilator guide of FIG. 15;

FIG. 17 is a front plan view of the dilator guide of FIG. 15;

FIG. 18 is a front sectional view of the dilator guide of FIG. 15, taken along line B-B in FIG. 16;

FIG. 19 is a perspective view of another example of a transducer probe forming part of the ultrasonic transducer probe system of FIG. 1;

FIG. 20 is a plan view of the transducer probe of FIG. 19;

FIG. 21 is a rear plan view of the transducer probe of FIG. 19;

FIG. 22 is a side sectional view of the transducer probe of FIG. 19;

FIGS. 23-24 are perspective views of another example of a transducer probe forming part of the ultrasonic transducer probe system of FIG. 1;

FIGS. 25-26 are front plan views of the transducer probe of FIG. 23;

FIG. 27 is a side plan view of the transducer probe of FIG. 23;

FIG. 28 is a perspective view of another example of a stabilizer tube forming part of the intraoperative ultrasound probe system of FIG. 1;

FIG. 29 is a top perspective view of the stabilizer tube of FIG. 28;

FIG. 30 is a top plan view of the stabilizer tube of FIG. 28;

FIG. 31 is a side plan view of the stabilizer tube of FIG. 28;

FIG. 32 is a side sectional view of the stabilizer tube of FIG. 28, taken along line C-C in FIG. 29;

FIGS. 33-34 are perspective views of the transducer probe of FIG. 23 coupled with the stabilizer tube of FIG. 28;

FIGS. 35-36 are perspective and top plan views, respectively, of the dilator guide of FIG. 15 coupled with the stabilizer tube of FIG. 28;

FIG. 37 is a plan view of one example of an electronic device including a computer and display unit forming part of the intraoperative ultrasound probe system of FIG. 1;

FIG. 38 is a block diagram illustrating an exemplary operating room setup for the method of using the intraoperative ultrasound probe system of FIG. 1 according to one embodiment;

FIGS. 39-43 are perspective views of various steps of the method of using the intraoperative ultrasound probe system of FIG. 1 according to one embodiment;

FIGS. 44-45 are examples of graphic user interface (GUI) screens forming part of the intraoperative ultrasound probe system of FIG. 1 according to one embodiment;

FIGS. 46-47 are perspective views of additional method steps;

FIG. 48 is a perspective view of an example of an echogenic surgical implant according to one embodiment;

FIGS. 49-50 are block diagrams of examples of computer systems forming part of the intraoperative ultrasound probe system of FIG. 1;

FIG. 51 is flowchart depicting an example method of generating a three-dimensional ultrasound image of intervening anatomy between a patient's dura and a surgical target site, according to some embodiments;

FIG. 52 is a perspective view of an example of a surgical robot coupled with an example ultrasound probe, according to some embodiments;

FIG. 53 is a block diagram depicting a top view of an example of a cannulated probe, according to some embodiments; and

FIG. 54 is a block diagram depicting a side view of the cannulated probe of FIG. 53, according to some embodiments.

### DETAILED DESCRIPTION OF A PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The intraoperative ultrasound probe system and related methods disclosed herein boasts a variety of inventive features and components that warrant patent protection, both individually and in combination.

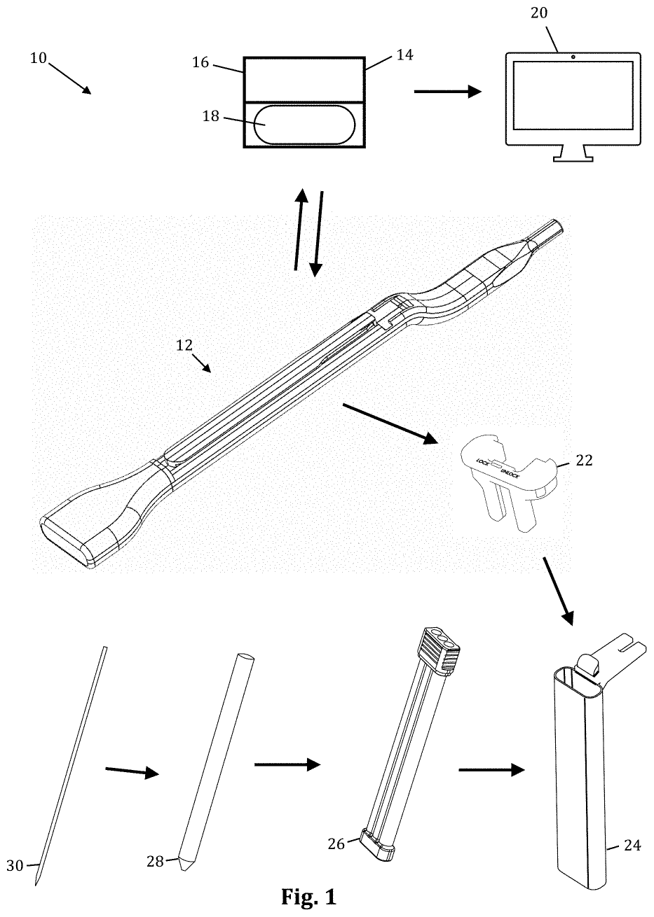

The present disclosure describes an intraoperative ultrasound probe system and related methods capable of using ultrasound imaging to help a surgeon visually determine a trajectory to safely place one or more instruments (e.g., needle, guide wire, dilator, cannula, etc.) through tissue (e.g., muscle, fat, brain, liver, lung, etc.) without damaging nearby neurovascular structures. FIG. 1 illustrates an example of an intraoperative ultrasound probe system 10 according to one embodiment of the present disclosure. By way of example, the intraoperative ultrasound probe system 10 includes a probe 12 configured for emitting and receiving ultrasound waves in electronic communication with an electronic device 14 (or computer 14) including a computer processor 16 for processing data received by the probe 12, software 18 for providing a set of executable instructions to the processor, and a display unit 20 (integrated or standalone) configured to display an ultrasound image based on the processed data. The electronic device 14 may be any stationary or portable computer system including a processor 16, software 18, and the ability to communicate with a display unit 20 (integrated or standalone), including but not limited to a laptop, desktop, workstation, personal digital assistant, server, blade server, mainframe, cellular telephone, smartphone, tablet computer, and/or other similar computing devices. The intraoperative ultrasound probe system 10 of the present disclosure further includes a variety of instruments and accessories, including but not limited to a transducer stabilizer 22, an stabilizer tube 24, dilator guide 26, dilator 28, and a guide wire (e.g. K-wire) 30.

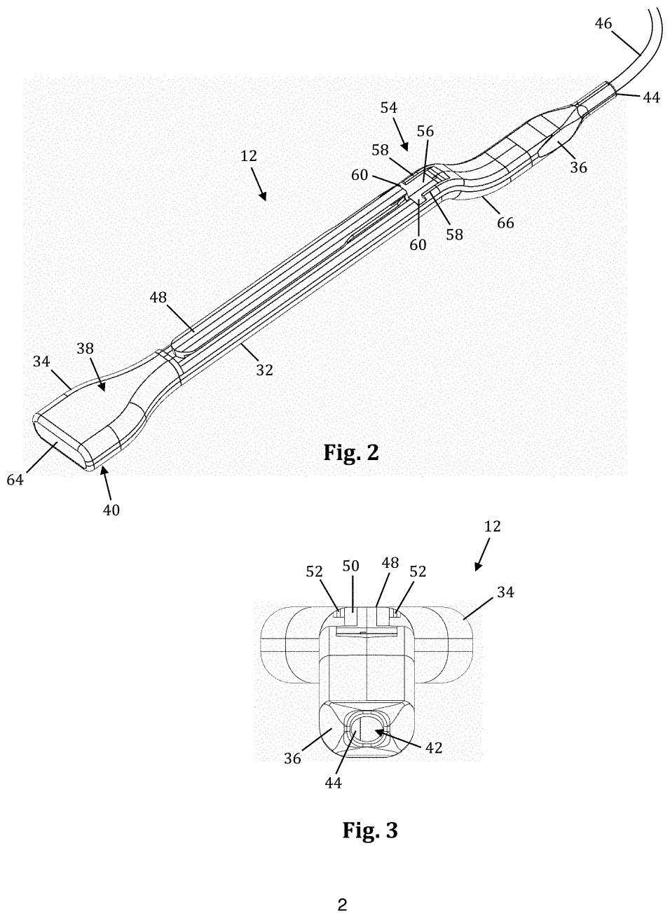

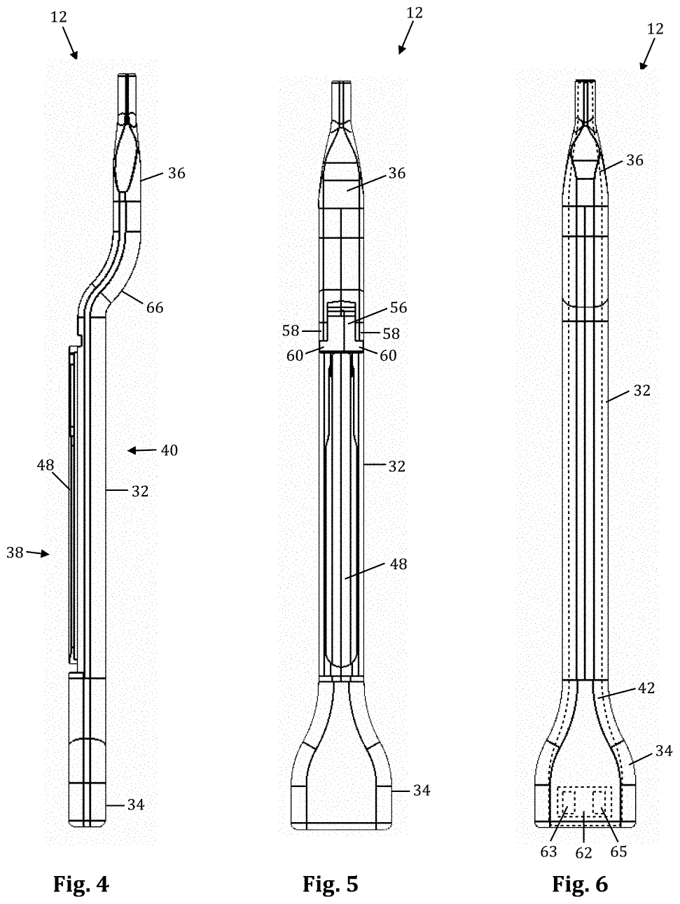

FIGS. 2-6 illustrate an example of an ultrasonic transducer probe 12 according to one embodiment of the disclosure. By way of example only, the probe 12 comprises an elongated housing member having an elongated main body portion 32, a distal end 34, a proximal end 36, a superior face 38, an inferior face 40, an inner cavity 42, and a proximal aperture 44 through which a communication cable 46 passes that may, by way of example only, connect the probe 12 to one or more of a power source, display device, computer, etc. The main body portion 32 includes an elongated coupling track 48 positioned on the superior face 38 that extends substantially the length of the main body portion 32. The coupling track 48 is configured to slidably couple with one or more attachments or accessories (not shown). By way of example, and as best shown in FIG. 3, the coupling track 48 comprises an elongated central beam 50 having a pair of elongated lateral flanges 52 creating an overhang such that the coupling track 48 has a generally “T”-shaped cross section (e.g. a “dove-tail” configuration).

The main body portion 32 further comprises a passive locking element 54 configured to engage one or more accessories, including but not limited to (and by way of example only) a cantilever locking element of a guide sleeve (not shown), and/or a portion of the transducer stabilizer 22 described below. By way of example only, the passive locking element 54 comprises a recess 56 formed within the superior face 38 near the proximal end of the coupling track 48. Formation of the recess 56 creates a pair of sidewalls 58 positioned on either side of the recess 56 and extending partially the length of the recess 56, leaving a gap 60 between the distal ends of the sidewalls 58 and the distal end of the recess 56.

The distal end 34 has an enlarged width (for example) to accommodate the ultrasonic transducer array 62 disposed within the interior cavity 42 at the distal end 34 of the probe 12. By way of example only, the transducer array 62 comprises at least one emission element 63 and at least one sensing element 65. The at least one emission element 63 may be configured to emit high-frequency sound pulses in a direction away from the distal end 34. At least some of the emitted high-frequency sound pulses may be reflected by boundaries between body tissues. The at least one sensing element 65 may be configured to receive the reflected sound pulses as radio frequency (RF) data, which is then transmitted to the processor 16 by way of the communication cable 46 (for example) or other suitable method of electronic communication (e.g. wired, wireless, WiFi, Bluetooth, etc.). The distal end 34 further comprises a leading face 64. By way of example, the leading face 64 may be generally planar with smooth rounded edges to minimize trauma to surrounding tissue as the probe 12 is advanced and retracted through the operative corridor.

The proximal end 36 includes curved portion 66 such that the proximal end 36 is laterally offset from the main body portion 32 in the inferior direction. By way of example, the superior and inferior faces 38, 40, respectively, are generally planar with smooth, rounded edges to minimize trauma to surrounding tissue as the probe 12 is advanced through the operative corridor.

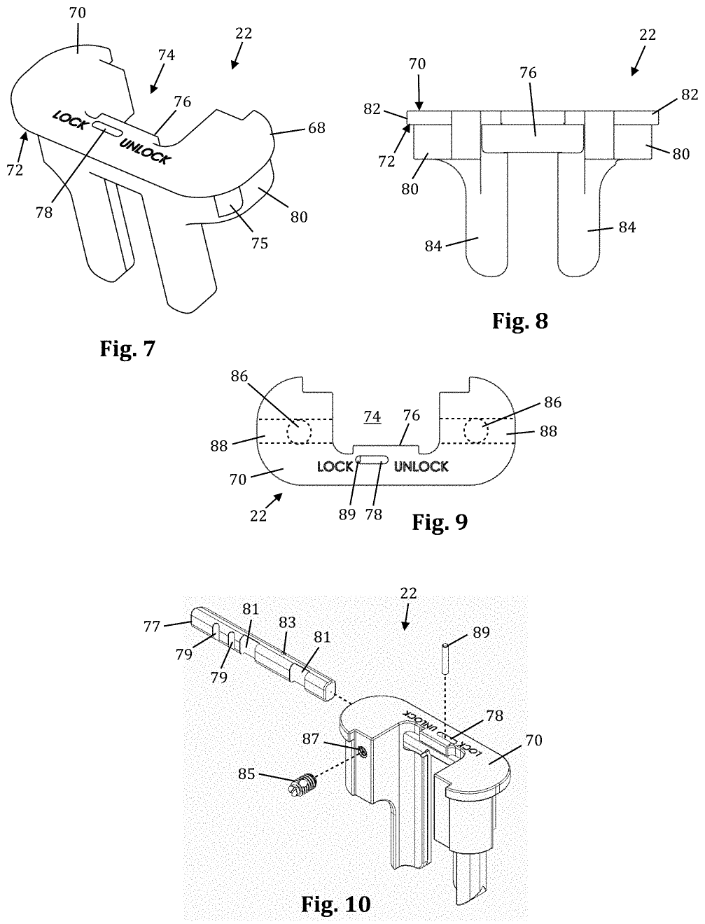

FIGS. 7-10 illustrate an example of a transducer stabilizer 22 configured for use with and forming part of the intraoperative ultrasound probe system 10 according to one embodiment of the disclosure. The distal region 34 of the probe 12 is sized and configured to slidingly engage the stabilizer tube 24 as described below. However, the proximal region 36, being of narrower width than the distal region 34, does not itself engage the stabilizer tube 24 and as a result would be free to move (and thereby change trajectory angle during use) absent a stabilizing intermediate structure. The transducer stabilizer 22 is configured to attach to the transducer probe 12 near the proximal region 36 and further engage the stabilizer tube 24 such that the narrower proximal region 36 of the probe 12 is secured in position when the probe is inserted into the stabilizer tube 24.

By way of example only, the transducer stabilizer 22 has a main body portion 68 comprising a superior surface 70, inferior surface 72, a central recess 74 formed therein that is open to one side of the stabilizer 22, and a lateral channel 75 extending through the main body portion. The superior surface 70 is generally planar with smooth rounded edges such that the stabilizer 22 has a generally rounded rectangle cross-sectional shape (see e.g. FIG. 9). The central recess 74 is sized and configured to receive a portion of the proximal region 36 of the probe 12 therein. The stabilizer 22 further includes a first engagement element 76 and a lock bar 77 configured to engage the probe 12 and couple the stabilizer 22 to the probe 12. By way of example, the engagement element 76 is positioned at the closed side of the recess (opposite the open side) and is configured to engage the passive locking element 54 (or similar feature) on the probe 12. The lock bar 77 is sized and configured to be received within the lateral channel 75 and includes a pair of locking recesses 79, a pair of engagement recesses 81, and an upper aperture 83. The locking recesses 79 are configured to receive therein a distal end of a lock screw 85 when the locking recess 79 is aligned with a threaded bore 87 formed within the main body portion 68. The engagement recesses 81 are configured to receive at least a portion of the sidewalls 58 therein when the transducer stabilizer 22 is coupled with the probe 12. The stabilizer 22 further includes a visual indicator window 78 that will indicate a positive locking engagement between the stabilizer 22 and probe 12. More specifically, the visual indicator window 78 includes an indicator pin 89 that is coupled to the lock bar 77 via the upper aperture 83. By way of example, the position of the indicator pin 89 relative to markings on the superior surface 70 of the may indicate to the user whether or not the stabilizer 22 is locked to the probe 12.

The transducer stabilizer 22 is further configured to securely engage the stabilizer tube 24 while simultaneously coupled to the probe 12. To facilitate this engagement, the stabilizer 22 further includes a pair of inferior buttresses 80 extending from the inferior surface 72 of the main body portion 68. The inferior buttresses 80 are located at either end of the stabilizer (e.g. one on each side of the central recess 74) and have a curved perimeter shape that corresponds to the perimeter shape of the interior lumen 96 of the stabilizer tube 24 such that the inferior buttresses 80 are sized and shaped to be snugly received within the interior lumen 80 of the stabilizer tube 24. The main body portion 68 (including the superior and inferior surfaces 70, 74) have a slightly larger perimeter than the buttresses 80 so as to create an overhang or lip 82 that prevents the entire stabilizer 22 from entering the interior lumen 96 of the stabilizer tube 24. A pair of elongated flanges 84 extend further inferiorly into the interior lumen 96 (when coupled to the stabilizer tube 24) to provide further stability. As a result, the transducer stabilizer 22 is configured to “sit” on top of the stabilizer tube 24 when engaged thereto and maintain the proximal end 46 of the probe 12 in a fixed orientation relative to the stabilizer tube 24.

The transducer stabilizer 22 may be manufactured from a medical-grade radiolucent material such as PEEK (poly-ether-ether-ketone), PEKK (poly-ether-ketone-ketone), etc. and may further contain radiographic markers 86, 88 positioned to signal the location of one or more of the guide channels 116 of the dilator guide 26 (described below), and by extension the location of possible access pathways, under fluoroscopy. By way of example, radiographic markers 86 may be spot markers to indicate the location of the top openings of the guide channels 116 of the dilator guide 26. The radiographic marker 88 may be a linear marker to indicate the alignment and/or angular orientation of the guide channels 116. In some embodiments, the transducer stabilizer may be made from anodized aluminum. In some embodiments, the probe 10 may include internal metallic structure that function as radiographic elements to indicate where the multiple guide channels 116 of the dilator guide 26 will be located once the dilator guide 26 is advanced into the stabilizer tube 24, enabling the surgeon to ensure that all of the potential guide channels 116 are also in alignment with the surgical target site 426.

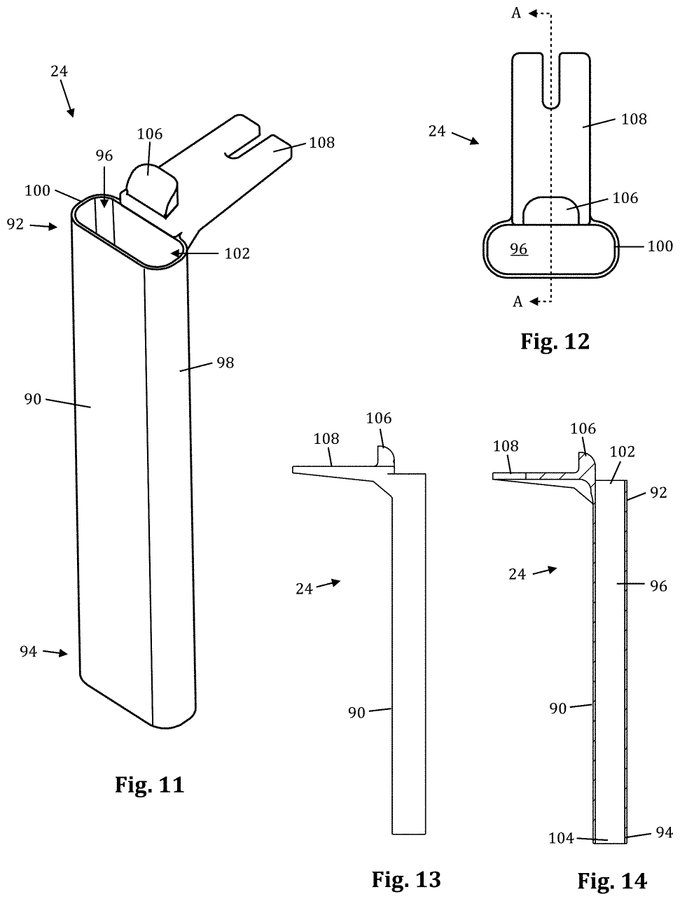

FIGS. 11-14 illustrate an example of a stabilizer tube 24 configured for use with the intraoperative ultrasound probe system 10 according to one embodiment of the disclosure. By way of example only, the stabilizer tube 24 comprises an elongated cannulated sleeve 90 having a proximal end 92, a distal end 94, and an interior lumen 96 extending from the proximal end 92 to the distal end 94. By way of example only, the sleeve 90 and the interior lumen 96 each have a generally rounded rectangle cross-sectional shape. The sleeve 90 has a smooth outer surface 98 to minimize trauma to surrounding tissue during use. The interior lumen 96 is sized and configured to receive at least a portion of the buttresses 80 therein at the proximal end 92 and to further receive the dilator guide 26 therein, described below. The proximal end 92 further includes a proximal rim 100 configure to engage the lip 82 of the stabilizer 22 to prevent the stabilizer 22 from fully entering the interior lumen 96. The sleeve 90 further comprises a proximal aperture 102 and a distal aperture 104 at the respective ends of the interior lumen 96 to enable ingress and egress of various surgical instruments through the stabilizer tube 24. The proximal end 92 further includes a superior flange 106 to buttress the laterally-offset curved portion 66 of the probe 12 and a laterally extending flange 108 configured to engage with an articulating arm (for example) to register the stabilizer tube 24 (and by extension any instruments associated with it such as the probe 12, dilator guide 26, etc.) to the patient's bedrail in a fixed orientation.

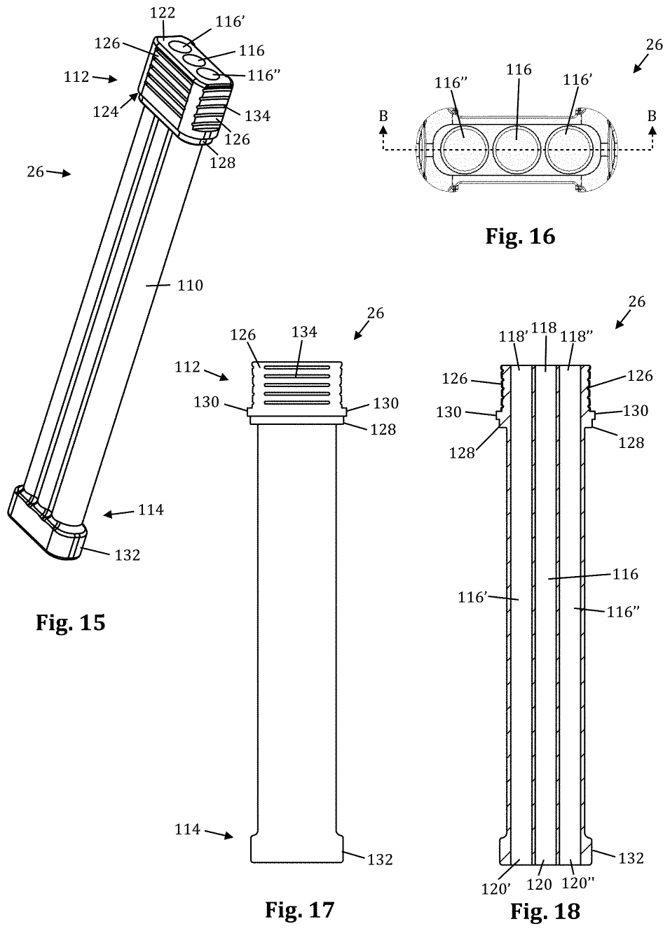

FIGS. 15-18 illustrate an example of a dilator guide 26 configured for use with the intraoperative ultrasound probe system 10 according to one embodiment of the disclosure. By way of example only, the dilator guide 26 comprises an elongated cannulated sleeve 110 having a proximal end 112, a distal end 114, and one or more guide channels 116 in the form of interior lumens extending from the proximal end 112 to the distal end 114 and having proximal openings 118 and distal openings 120 to allow ingress and egress of various instrumentation therethrough. The dilator guide 26 shown and described herein by way of example only has three cylindrical guide channels, including a central guide channel 116 and a pair of lateral guide channels 116′, 116″. However, it should be understood that the dilator guide 26 may be provided with any number of guide channels 116 without departing from the scope of the disclosure. As will be explained below, multiple guide channels 116 allow a user to examine multiple potential approach trajectories at the same time, while also providing the user the ability to select and employ any one of the examined trajectories without moving the stabilizer tube 24 (and by extension any instrumentation associated therewith). The cannulated sleeve 110 has a smooth outer surface to minimize trauma to surrounding tissue during use. The guide channels 116, 116′, 116″ are each sized and configured to receive at least one dilator therethough, however any instrument having a diameter or width smaller than the diameter of the guide channels may pass through.

The proximal end 112 includes a superior surface 122, an inferior surface 124, and a plurality of sidewalls 126 extending between the superior and inferior surfaces 122, 124. The superior surface is generally planar, has a rounded rectangle perimeter shape, and includes a plurality of apertures 118 (e.g. proximal guide channel apertures 118, 118′, 118″) formed therein. The inferior surface 124 includes an inferior buttress 128 extending inferiorly therefrom, the inferior buttress 128 having a perimeter sized and shaped to correspond to the perimeter shape of the interior lumen 96 of the stabilizer tube 24, such that the inferior buttress 128 is snugly received within the interior lumen 96 of the stabilizer tube 24. The inferior surface 124 has a slightly larger perimeter than the buttress 128 so as to create an overhang or lip 130 that prevents the proximal end 112 of the dilator guide 26 from entering the interior lumen 96 of the stabilizer tube 24. As a result, the proximal end 112 is configured to “sit” on top of the stabilizer tube 24 when engaged thereto and maintain the dilator guide 26 (and importantly, the guide channels 116 in a fixed orientation relative to the stabilizer tube 24. Similarly, the distal end 114 of the dilator guide 26 has a perimeter surface 132 having a size and shape corresponding to the perimeter size and shape of the interior lumen 96 (when coupled with the stabilizer tube 24) so that the distal end 114 is snugly received within the interior lumen 96 to provide further stability.

The sidewalls 126 form the outer perimeter of the proximal end 112 and include a plurality of friction elements 134 (e.g. ridges, knobs, surface roughening, and the like) dispersed thereupon. The friction elements 134 enable a user to grab hold of and exert pulling force on the dilator guide 26 to remove the dilator guide 26 from the stabilizer tube 24 after use.

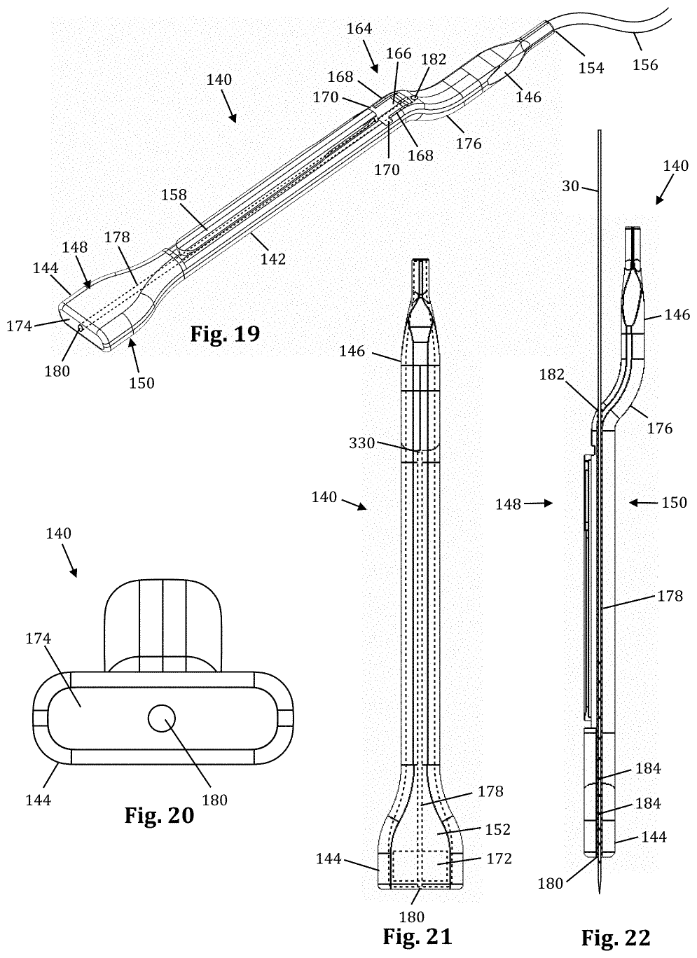

FIGS. 19-22 illustrate another example of a probe 140 forming part of the intraoperative ultrasound probe system 10 of the present disclosure. As will be explained below, the probe 140 of the present example may be cannulated to enable advancement of the probe 140 along a surgical guide wire (e.g. K-wire 30). By way of example, the probe 140 comprises an elongated housing member having an elongated main body portion 142, a distal end 144, a proximal end 146, a superior face 148, an inferior face 150, an inner cavity 152, and a proximal aperture 154 through which a communication cable 156 passes that may, by way of example only, connect the probe 140 to one or more of a power source, display device, computer, etc. In some embodiments, the main body portion 142 may include an elongated coupling track 158 positioned on the superior face 148 that extends substantially the length of the main body portion 142, the coupling track 158 having a similar structure and function to the coupling track 48 described above. The main body portion 142 further comprises a passive locking element 164 configured to engage one or more accessories, including but not limited to (and by way of example only) a portion of the transducer stabilizer 22 described above. By way of example only, the passive locking element 164 comprises a superior recess 166 formed within the superior face 148 near the proximal end 146. Formation of the recess 166 creates a pair of sidewalls 168 positioned on either side of the recess 166 and extending partially the length of the recess 166, leaving a gap 170 between the distal ends of the sidewalls 168 and the distal end of the recess 166.

The distal end 144 has an enlarged width (for example) to accommodate the ultrasonic transducer array 172 disposed within the distal end 144. By way of example only, the transducer array 172 comprises at least one emission element and a least one sensing element. The at least one emission element may be configured to emit high-frequency sound pulses in a direction away from the distal end 144. At least some of the emitted high-frequency sound pulses may be reflected by boundaries between body tissues. The at least one sensing element may be configured to receive the reflected sound pulses as radio frequency (RF) data, which is then transmitted to the processor 16 by way of the communication cable 156 (for example) or other suitable method of electronic communication (e.g. wired, wireless, WiFi, Bluetooth, etc.). The distal end 144 further comprises a leading face 174. By way of example, the leading face 174 may be generally planar with smooth rounded edges to minimize trauma to surrounding tissue as the probe 140 is advanced and retracted through the operative corridor. The proximal end 146 may include a curved portion 176 such that the proximal end 146 is laterally offset from the main body portion 142 in the inferior direction. By way of example, the superior and inferior faces 148, 150, respectively, are generally planar with smooth, rounded edges to minimize trauma to surrounding tissue as the probe 140 is advanced through the operative corridor.

By way of example, the probe 140 may be cannulated such that the probe 140 comprises an interior corridor 178 extending substantially the length of the main body portion 142 and configured to allow passage of one or more surgical instruments (e.g. dilator 28, K-wire 30) therethrough. The distal end 144 of the probe 140 includes a distal aperture 180 formed within the leading face 174, the distal aperture 180 comprising the distal terminus of the interior corridor 178 and allowing ingress into and/or egress from the interior corridor 178. The proximal terminus of the interior corridor 178 comprises a proximal aperture 182 positioned distally of the curved portion 176. The proximal aperture 182 comprises the proximal terminus of the interior corridor 178 and allows ingress into and/or egress from the interior corridor 178. The K-wire 30 may include a series of echogenic elements 184 (e.g. notches, ridges, etc.) configured to reflect sound waves to make the K-wire 30 “visible” during ultrasound imaging.

By way of example only, the interior corridor 178 may occupy space within the inner cavity 152 of the probe 140, but is physically separate from the inner cavity 152 to ensure that no foreign material (e.g. patient tissue, etc.) may penetrate the inner cavity 152. Thus, internal probe elements (e.g. transducer array 172, communication cable 156, etc.) may be positioned within the inner cavity 152 around the interior corridor 178. Providing the probe 140 with a cannulation (e.g. interior corridor 178) enables an instrument (e.g. dilator 28, K-wire 30) to be advanced directly through the probe 140 without the need for a separate insertion corridor (e.g. dilator guide 26), which may be advantageous in some surgical situations in that the instrument is inserted directly through the field of view as opposed to alongside the field of view.

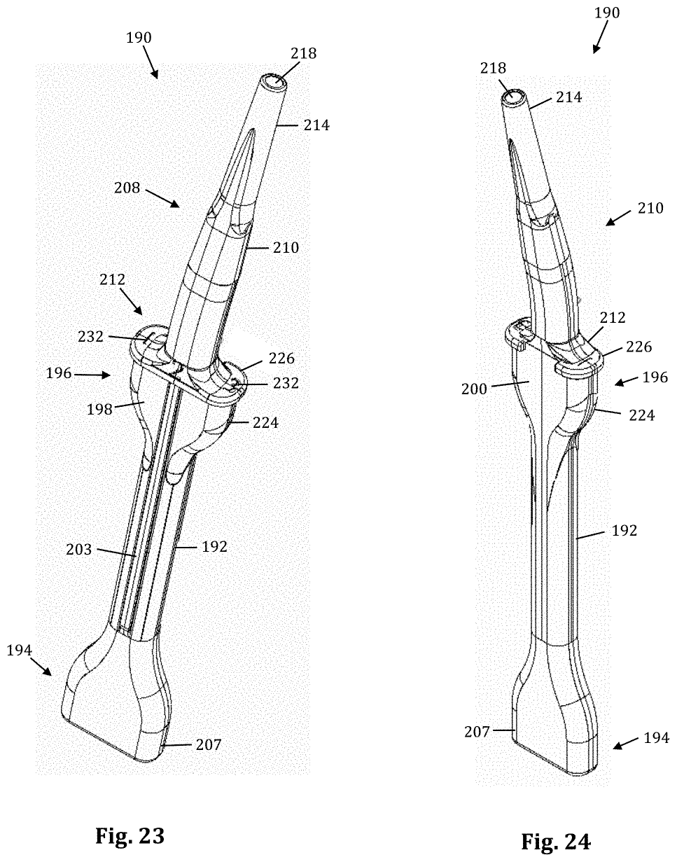

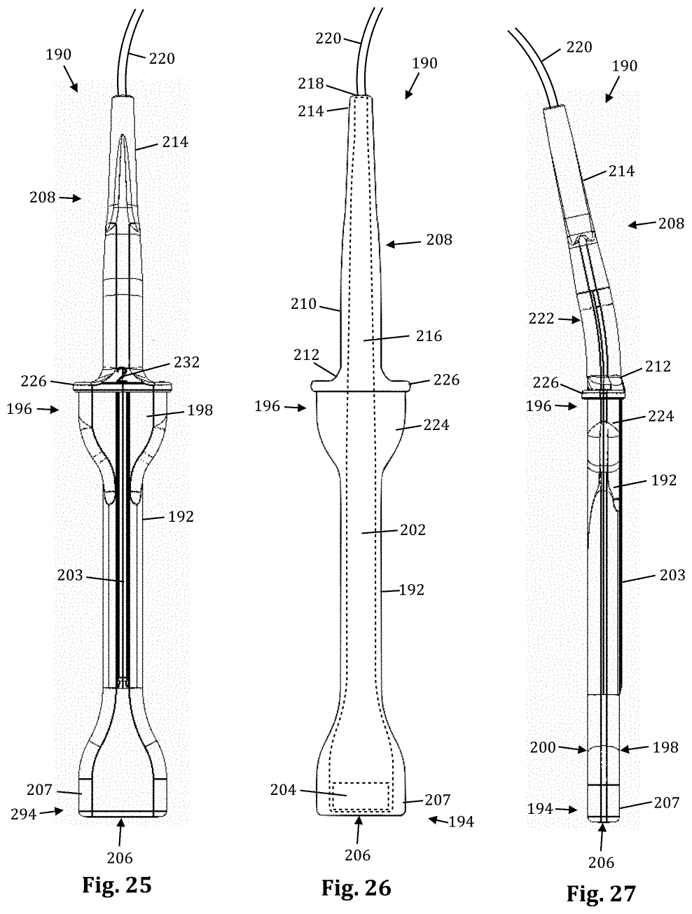

FIGS. 23-27 illustrate another example of a probe 190 configured for use with (and forming part of) the intraoperative ultrasound probe system 10 of the present disclosure. For the purpose of illustration, the probe 190 of the present example embodiment will be described for use with the stabilizer tube 240 described below, however it should be understood that the probe 190 may also be used with stabilizer tube 24 described above. By way of example only, the probe 190 comprises an elongated housing member 192, having a distal end 194, a proximal end 196, a superior face 198, an inferior face 200, and an inner cavity 202 extending therethrough. The elongated housing member 192 may have generally smooth planar surfaces (e.g. comprising the superior face 198 and inferior face 200) and smooth rounded edges to minimize trauma to surrounding patient tissue during use. By way of example, elongated housing member 192 includes a coupling track 203 positioned on the superior face 198 that extends substantially the length of the housing member 192. The coupling track 203 comprises an elongated beam element (by way of example) configured to slidably couple with one or more attachments or accessories, including but not limited to (and by way of example only) the stabilizer tube 240 described below (See, e.g. FIG. 34).

The distal end 194 has an enlarged width (for example) to accommodate an ultrasonic transducer array 204 (e.g. including at least one emission element and at least one sensing element) disposed within the interior cavity 202 at the distal end 194 of the probe 190. The distal end 194 further comprises a leading face 206. By way of example, the leading face 206 may be generally planar with smooth rounded edges to minimize trauma to surrounding tissue as the probe 190 is advanced and retracted through the operative corridor. By way of example only, the distal end 194 of the elongated housing member 192 comprises an outer surface 207 having a curved perimeter shape that corresponds to the perimeter shape of the interior lumen 248 of the stabilizer tube 240 (and/or interior lumen 96 of stabilizer tube 24). This shape encourages a snug interaction between the distal end 194 and the stabilizer tube 240 to minimize or eliminate non-translational movement of the distal end 194 relative to the stabilizer tube 240 during use. By way of example only, the proximal end 196 of the elongated housing member 192 may also have an enlarged width, giving the elongated housing member 192 a generally hourglass shape.

The probe 190 further comprises a proximal extension 208 extending proximally from the proximal end 196 of the elongated housing member 192. The proximal extension 208 comprises an elongated body 210 having a distal end 212, a proximal end 214, and an inner cavity 216 extending therethrough from the distal end 212 to the proximal end 214. The distal portion of the inner cavity 216 is continuous with the inner cavity 202 of the elongated housing member 192. The proximal end 214 further comprises a proximal aperture 218 through which a communication cable 220 passes that may, by way of example only, connect the probe 190 (e.g. including but not limited to the transducer array 204) to one or more of a power source (not shown), display device 20, computer 14, etc.

By way of example, the proximal extension 208 includes a curved portion 222 configured such that the proximal end 214 is laterally offset from the distal end 212 and thus the elongated housing member 192 in the inferior direction.

By way of example, the probe 190 is configured to securely engage the stabilizer tube 240 and/or stabilizer tube 24 without the need for additional accessories. As previously mentioned, the proximal end 196 of the elongated housing member 192 may have an enlarged width, giving the elongated housing member 192 a generally hourglass shape. More specifically, the proximal end 196 of the elongated housing member 192 comprises an outer surface 224 having a curved perimeter shape that corresponds to the perimeter shape of the interior lumen 248 of the stabilizer tube 240 such that the proximal end 196 is sized and shaped to be snugly received within the interior lumen 248 of the stabilizer tube 240. This shape encourages a snug interaction between the proximal end 196 and the stabilizer tube 240 to minimize or eliminate non-translational movement of the proximal end 196 relative to the stabilizer tube 240 during use of the probe 190. The distal end 212 of the proximal extension 208 may have a slightly larger perimeter than the proximal end 196 of the elongated housing member 192 so as to create an overhang or lip 226 that prevents the proximal extension 208 from entering the interior lumen 248 of the stabilizer tube 240. As a result, the proximal extension 208 is configured to “sit” on top of the stabilizer tube 240 when engaged thereto to provide a hard stop for advancement of the probe 190 through the stabilizer tube 240 and also maintain the proximal end 196 of the probe 190 in a fixed orientation relative to the stabilizer tube 240.

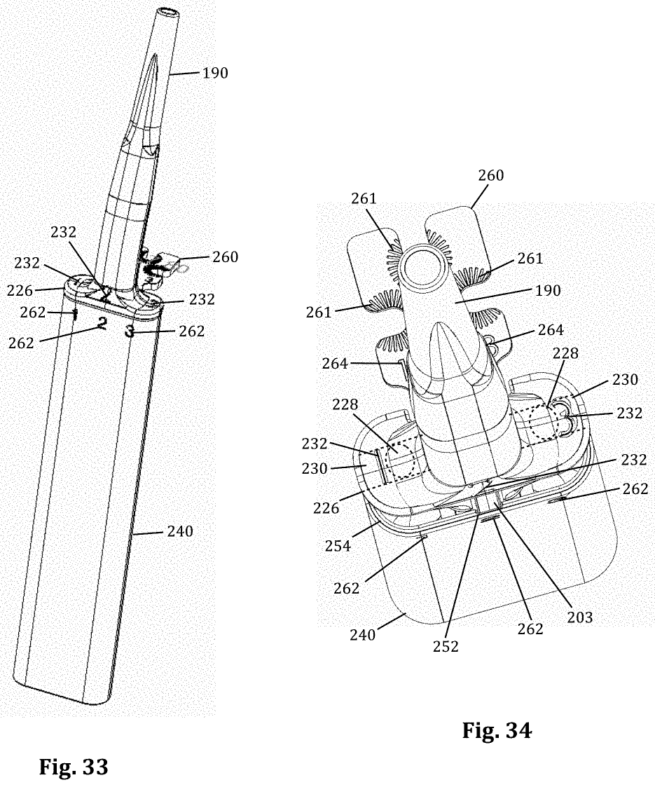

By way of example, the distal end 212 of the proximal extension 208 may further contain radiographic markers 228, 230 (see, e.g., FIG. 34) positioned to signal the location of one or more of the guide channels 116 of the dilator guide 26, and by extension the location of possible access pathways, under fluoroscopy. By way of example, radiographic markers 228 may be spot markers to indicate the location of the top openings of the guide channels 116 of the dilator guide 26. The radiographic marker 230 may be a linear marker (or markers) to indicate the alignment and/or angular orientation of the guide channels 116. In some embodiments, the distal end 212 of the proximal extension 208 may further include (by way of example only) one or more surface markings 232 configured to indicate and identify the future positions of the guide channels 116 of the dilator guide 26, once the probe 190 is removed from the stabilizer tube 240 and replaced with the dilator guide 26 as described below. By way of example, the surface markings 232 may comprise a numerical indication correlating to a specific guide channel 116 (e.g. a “1” to indicate the position of guide channel 116, a “2” to indicate the position of guide channel 116′, a “3” to indicate the position of guide channel 116″, and so forth). This relationship is illustrated by way of example in FIGS. 33-36.

In some embodiments, the probe 190 may be cannulated such that the probe 190 comprises an interior corridor (not shown) extending substantially the length of the elongated housing member 192 and configured to allow passage of one or more surgical instruments (e.g. K-wire 30) therethrough.

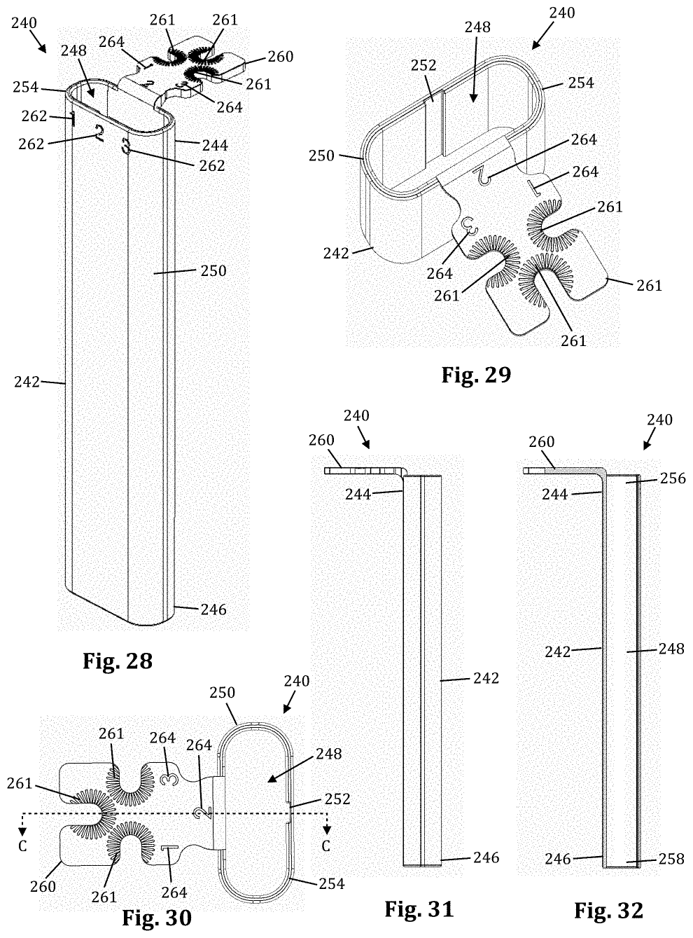

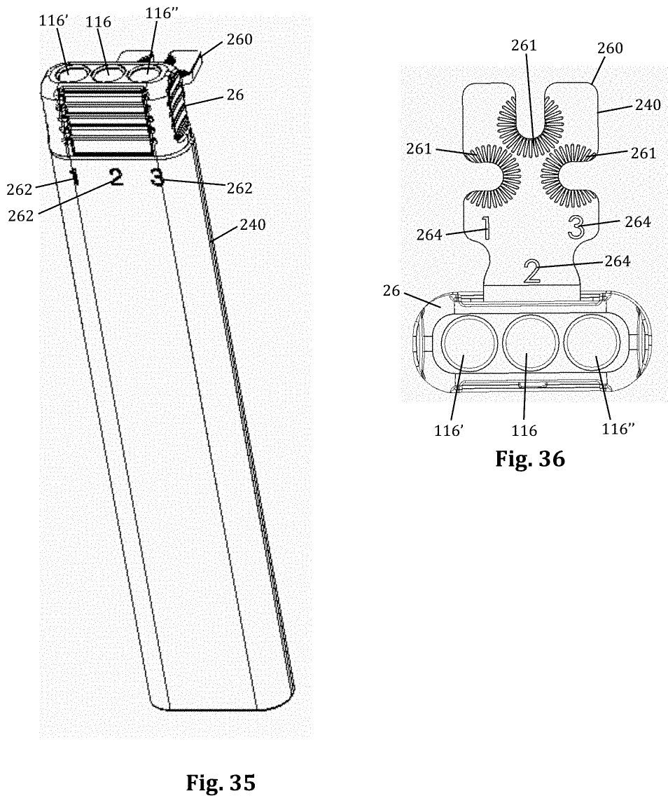

FIGS. 28-32 illustrate an example of a stabilizer tube 240 configured for use with the intraoperative ultrasound probe system 10 according to one embodiment of the disclosure. By way of example only, the stabilizer tube 240 comprises an elongated cannulated sleeve 242 having a proximal end 244, a distal end 246, and an interior lumen 248 extending from the proximal end 244 to the distal end 246. By way of example only, the sleeve 242 and the interior lumen 248 each have a generally rounded rectangle cross-sectional shape. The sleeve 242 has a smooth outer surface 250 to minimize trauma to surrounding tissue during use. The interior lumen 248 is sized and configured to snugly receive the enlarged-width distal end 194 and/or enlarged-width proximal end 196 of probe 190 (and/or the enlarged-width distal end 34 of the probe 12 and/or at least a portion of the inferior buttresses 80 of the transducer stabilizer 22) therein and to further receive the dilator guide 26 therein, as shown for example in FIGS. 35-36. The interior lumen further includes a longitudinally oriented elongated guide recess 252 configured to receive the coupling track 203 of the probe 190 therein to ensure the orientation of the probe 190 is maintained during use. The proximal end 244 further includes a proximal rim 254 configured to engage the lip 226 of the probe 190 to prevent the probe 190 from fully entering the interior lumen 248. The sleeve 242 further comprises a proximal aperture 256 and a distal aperture 258 at the respective ends of the interior lumen 248 to enable ingress and egress of various surgical instruments through the stabilizer tube 240. The proximal end 244 further includes laterally extending flange 260 including one or more attachment elements 261 configured to engage with an articulating arm (for example) to register the stabilizer tube 240 (and by extension any instruments associated with it such as the probe 190, dilator guide 26, etc.) to the patient's bedrail in a fixed orientation.

In some embodiments, the proximal end 244 of stabilizer tube 240 may further include (by way of example only) one or more surface markings 262 configured to indicate and identify the positions of the guide channels 116 of the dilator guide 26 irrespective of the instrument in use with the stabilizer tube 240 (e.g. probe 190, dilator guide 26, etc.). By way of example, the surface markings 262 may match the surface markings 232 on the probe 190 and comprise a numerical indication correlating to a specific guide channel 116 (e.g. a “1” to indicate the position of guide channel 116, a “20” to indicate the position of guide channel 116′, a “3” to indicate the position of guide channel 116″, and so forth). Similarly, the laterally extending flange 260 of the stabilizer tube 240 may further include (by way of example only) one or more surface markings 264 configured to indicate and identify the positions of the guide channels 116 of the dilator guide 26 irrespective of the instrument in use with the stabilizer tube 240 (e.g. probe 190, dilator guide 26, etc.). By way of example, the surface markings 264 may match the surface markings 232 on the probe 190 and surface markings 262 on the proximal end 244 and comprise a numerical indication correlating to a specific guide channel 116 (e.g. a “1” to indicate the position of guide channel 116, a “2” to indicate the position of guide channel 116′, a “3” to indicate the position of guide channel 116″, and so forth). This relationship is illustrated by way of example in FIGS. 33-36.

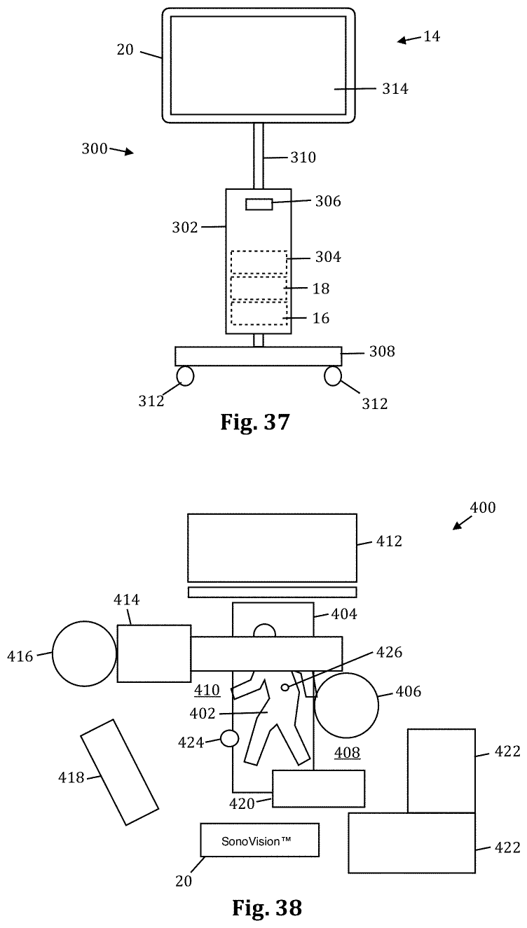

FIG. 37 illustrates one example of an electronic device 14 suitable for placement in an operating room (“O.R.”) and configured for use with the intraoperative ultrasound probe system 10 according to one embodiment of the disclosure. The electronic device 14 of the present example comprises a moveable unit 300 having a computer housing 302 (e.g. comprising a processor 16, software 18, data storage module 304, and a communications module 306 configured for wired and/or wireless communication with the probe 12), a base unit 308, and a display unit 20 coupled to a vertical displacement element 310. By way of example only, the base unit 308 is generally parallel to the floor, and has a plurality of wheel elements 312 (e.g. castors, etc.) that enable a user to move the moveable unit 300 to any desired position in the room. The vertical displacement element 310 may comprise any suitable structure capable of securely maintaining the display unit 20 at a useable height including but not limited to (and by way of example only) a pole, post, scaffolding, ladder, etc.). Although not shown, the electronic device 14 including the computer housing 302 and display unit 20 may be connected to a power source, either integrated with the electronic device or connected to A/C power via a power cord. In one embodiment, the electronic device 14 maybe A/C capable with a battery backup (not shown). The data storage module 304 may include internal storage or external storage. By way of example, the display unit 20 may have a screen 314 comprising a touch-screen interface that enables the user to provide instructions to the computer by selecting buttons or icons that the computer presents on the screen.

FIGS. 38-47 illustrate several steps of an exemplary method of using the intraoperative ultrasound probe system 10 of the present disclosure to safely determine an access trajectory to a surgical target site. By way of example only, the method is described herein in conjunction with establishing a lateral access trajectory through a psoas muscle to a surgical target site comprising an intervertebral disc space. However, it should be understood that the method described herein of using ultrasound imaging to identify and locate specific tissue types and determine a safe trajectory to a surgical target site may be used in any surgical situation.

The first step of the exemplary method is to position the display unit 20 of the electronic device 14 within the primary user's field of vision within the O.R, but outside the sterile field. Typically, the primary user is the surgeon performing the surgery on the patient. FIG. 38 is a block diagram illustrating an exemplary O.R. setup 400 for the method described herein. By way of example, the patient 402 is laying on an operating table or bed 404 and positioned on his/her side. The surgeon 406 is positioned on the posterior side 408 of the patient 402. Generally, O.R. setup 400 further comprises anesthesia apparatus and personnel 412 positioned at or near the head of the operating table 404, a C-arm 414 and C-arm technician 416 positioned on the anterior side 410 of the patient 402, a C-arm display 418, a mayo stand 420, and one or more back tables 422. As shown in FIG. 38, the optimal location for the display unit 20 (e.g. SonoVision™) is at the foot of the patient's bed 404, and also near the C-arm display 418 so that the surgeon can see both the fluoroscopic images (on the C-arm display 418) and the ultrasound images (on the display unit 20) at the same time. The next step is to connect an articulating arm 424 (e.g. Metrx flexible arm or equivalent) to the patient's operating table or bed 404 (e.g. on a rail or similar structure). The attachment point of the articulating arm 424 should be positioned anterior 410 and caudal (e.g. toward the patient's feet) of the surgical target site 426.

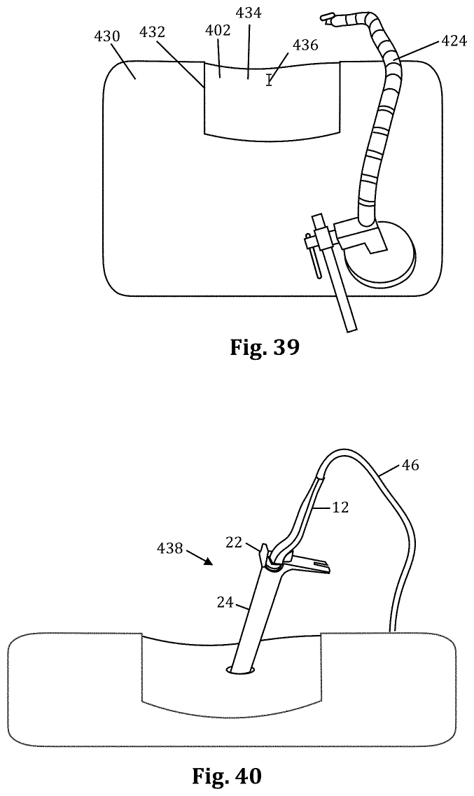

FIG. 39 illustrates a portion of the patient's body 402 covered by a surgical drape 430 having a window 432 exposing the area of the patient's skin 434 through which an access corridor must be formed to access the surgical target site 426. Once the equipment is set up in the correct spot, the next step is to identify the surgical target site 426 (e.g. vertebral level) with fluoroscopy and place a marking 436 on the patient's skin 434. After the skin has been marked, an initial incision is made on the marking 436. The surgeon may then use one or more fingers and/or blunt tissue dissection instruments to palpate the patient's tissue posterior to the peritoneum and down to the superficial psoas muscle.

At this point the probe 12 (or probe 190 and/or any probe described herein) may be connected to the electronic device 14, for example by connecting cable 46 of the probe 12 to the communications module 306 of the electronic device 14. By way of example only, the connecting cable 46 may have a connector element that is securely received within a port on the electronic device 14, the port being in electronic communication with the communications module 306. In one embodiment, the port may have a locking feature to positively lock the connector element within the port.

Once the probe 12 has been connected to the electronic device 14, the probe 12 and stabilizer tube 24 may be coupled in preparation for insertion into the patient through the incision. To accomplish this, the transducer stabilizer 22 may be coupled to the probe 12 in the manner described above, and the probe 12 with coupled stabilizer 22 may be inserted into the interior lumen 96 of the stabilizer tube 24, for example such that the buttresses 80 of the stabilizer 22 are received within the interior lumen 96 of the stabilizer tube 24. The leading face 64 of the probe 12 should be in alignment with (or very nearly in alignment with) the distal aperture 104 to ensure as smooth a leading surface as possible during advancement through the patient's tissue.

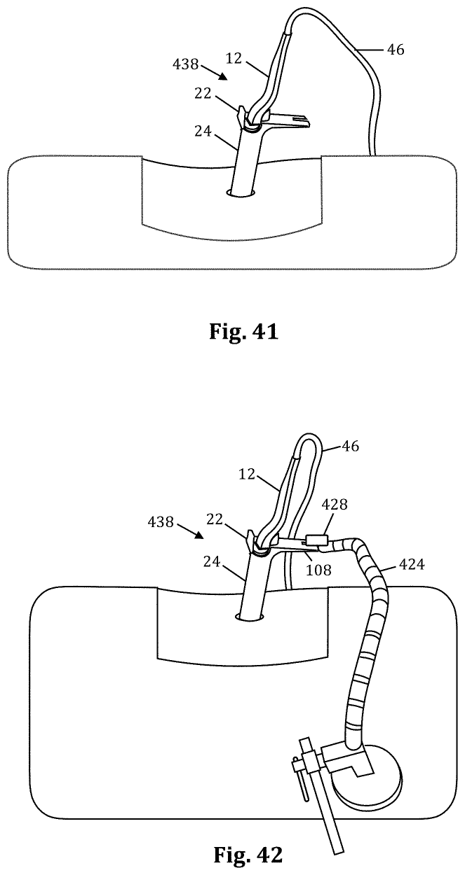

As shown in FIG. 40, the probe 12/stabilizer 22/conduit 24 assembly (hereinafter “probe assembly 438”) may be oriented parallel to the incision and then carefully advanced through the incision and fascia until the distal end 94 of the stabilizer tube 24, and leading face 64 of the probe 12 reach the surface of the external oblique muscle. At this point, the probe assembly 438 may be turned 90° (clockwise or counterclockwise) so that the probe assembly 438 is perpendicular to the incision and thus parallel to the muscle fibers of the external oblique muscle. After rotation is complete, the probe assembly 438 is then further advanced through the external oblique muscle. Once the distal end 94 of the stabilizer tube 24, and leading face 64 of the probe 12 exit the external oblique muscle and advance into the retroperitoneal space, the probe assembly 438 may be rotated 90° back to its original orientation parallel to the incision and also parallel to the target intervertebral disc space (see e.g. FIG. 41). The assembly is then positioned so that the leading face 64 of the probe 12 is resting on the superficial surface of the psoas muscle.

Once the leading face of the probe 64 is resting on the superficial psoas muscle, the probe assembly 438 may be registered to the operating table or bed 404 via a coupling with the articulating arm 424. As shown in FIG. 42, this may be accomplished by coupling a connecting element 428 on the distal end of the articulating arm 424 to the laterally extending flange 108 of the stabilizer tube 24. This connection ensures that the stabilizer tube 24, and any instrument securely associated therewith (e.g. including but not limited to probe 12, dilator guide 26, dilator 28, or K-wire 30), remain locked in position relative to the surgical target site.

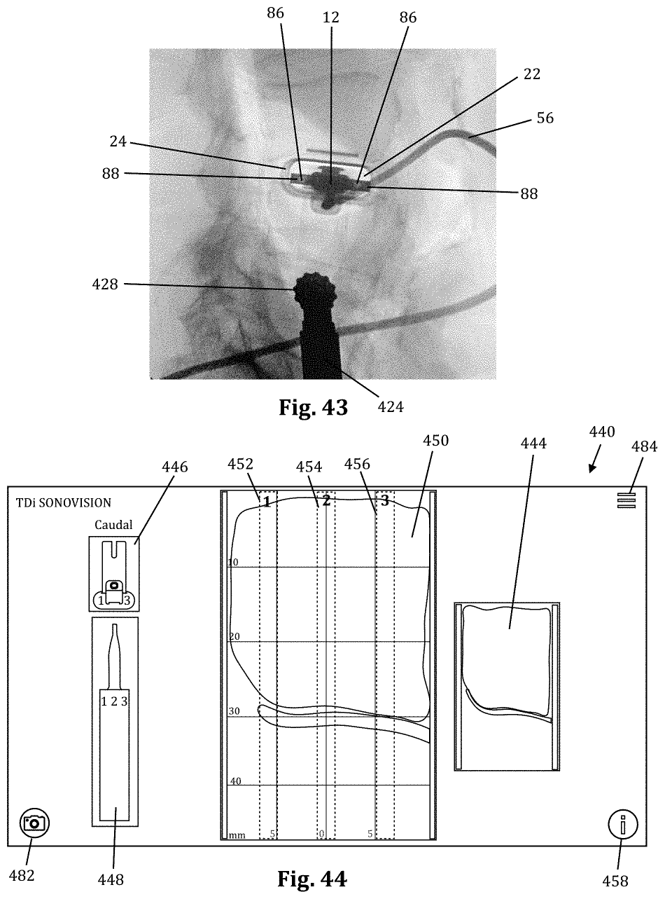

Referring to FIG. 43, the next step is to use the fluoroscopic imaging of the C-arm 414 to position the probe 12 over the disc space. Radiographic elements located on the probe 10 and/or stabilizer 22 may indicate where the multiple guide channels 116 of the dilator guide 26 will be located once the dilator guide 26 is advanced into the stabilizer tube 24, enabling the surgeon to ensure that all of the potential guide channels 116 are also in alignment with the surgical target site 426. By way of example only, such radiographic elements may include radiographic markers 86, 88 on the stabilizer 22, radiographic markers 228, 230 on the probe 190, and/or internal metallic structure of the probe 10 or probe 190.

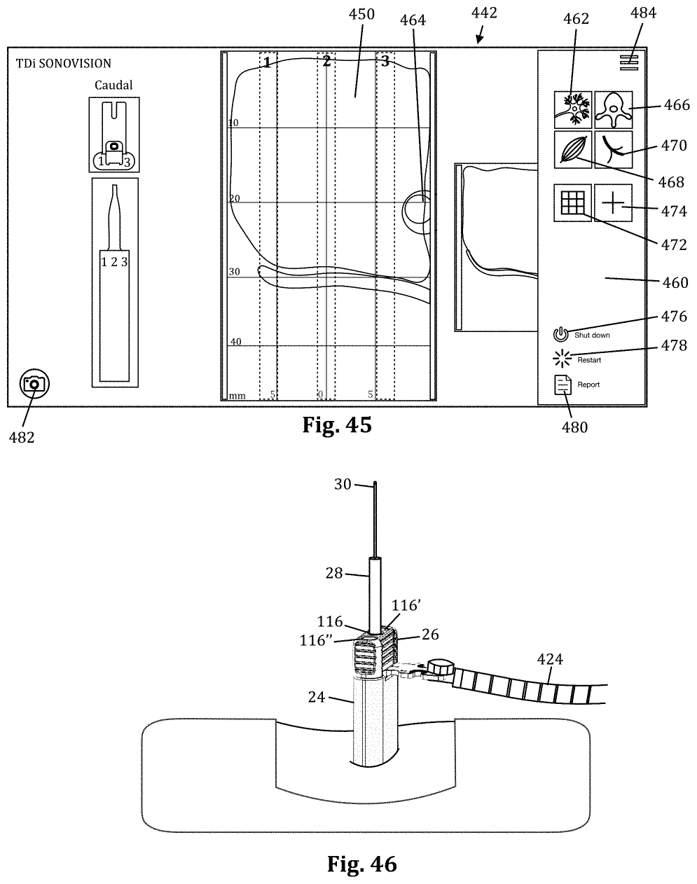

The user may now use the software 18 of the intraoperative ultrasound probe system 10 to determine if any of the available pathways through the psoas (as determined by the positioning of the radiographic markers 86 on the stabilizer 22) are clear of nerves and/or vasculature, and are therefore suitable for dilator advancement. By way of example only, FIGS. 44-45 illustrate example graphic user interface (GUI) screens 440, 442 that the electronic device 14 presents on the display unit 20 and a user encounters while using the intraoperative ultrasound probe system 10 according to one embodiment of the disclosure. By way of example, the GUI screen 440 of FIG. 44 may have three main sections. For example, the standard B-mode ultrasound image 444 is displayed on the right side of the screen. The section on the left displays a top view 446 and front plan view 448 of the probe assembly 438 currently in use, with numbers (e.g. 1, 2, 3) visible that correspond to the guide channel 116′, 116, 116″ (in the present example) of the dilator guide 26 to be used. The middle section presents the B-mode overlay 450 of the psoas muscle, which the computer displays in color and also includes the approximate available pathways through the psoas muscle based on the dilator guide 26 to be used and the current positioning of the probe assembly 438. In the present example, the first displayed pathway 452 corresponds to position “1” on the image on the left section of the GUI 440, which corresponds to guide channel 116′ of dilator guide 26. The second displayed pathway 454 corresponds to position “2” on the image on the left section of the GUI 440, which corresponds to guide channel 116 of dilator guide 26. The third displayed pathway 456 corresponds to position “3” on the image on the left section of the GUI 440, which corresponds to guide channel 116″ of dilator guide 26.

At this point the user may tap on the “circle-I” icon 458 in the lower right corner of the GUI screen 440 to direct the computer to present a pop-up menu 460, shown on GUI screen 442 in FIG. 45. The pop-up menu 460 includes a number of icons that the user may tap on to instruct the computer to display certain information on the B-mode overlay 450. For example, as shown in FIG. 45 the user may select a “nerve” icon 462, which instructs the computer to display location and proximity information of any nerves in the psoas muscle within the view of the probe 12. The computer will then display any such indication as color-coded shapes (e.g. circles 464 as shown in FIG. 45) and also indicate the unsafe area surrounding the nerve that should be avoided. By way of example, the displayed nerve may be up to 120% or more of identified size to build in a safety margin. Similar icons that instruct the computer to display similar information regarding other structures may be presented as well. For example, the GUI 442 of the present example includes a “bone” icon 466, “muscle” icon 468, “Doppler” icon 470, “Grid” icon 472, and an “Other” icon 474 for additional but perhaps less-used options. The displayed information may be displayed until the user unselects the information by tapping on the icon a second time. Additionally, the user may instruct the computer to display multiple sets of information at the same time by selecting several icons (e.g. nerve 462 and bone 466, etc.).

The pop-up menu 460 may further include a “shut down” icon 476 that when tapped by a user instructs the computer to begin the shutdown process, a “restart” icon 478 that when tapped by a user instructs the computer to restart the system, and a “Report” icon 480 that when selected by a user instructs the system to generate and store a session report to provide a record of the system events during the surgery. Each of the GUI screens 440, 442 (and any others) may also include a “camera” icon 482 that when selected by the user instructs the computer to capture and store a screenshot image, and a pull-down menu icon 484 that when selected by a user instructs the computer to present a pull-down menu that may present addition options for the user (for example including but not limited to login, surgery information, patient information, etc.)

If one or more of the indicated potential pathways 452, 454, 456 are determined to be clear of nerves, vasculature, and/or other structure to avoid and are therefore suitable for dilator advancement through the psoas, the guide number (e.g. 1, 2, 3, etc.) is noted for later use. If no pathway is determined to be suitably clear, then the probe assembly 438 may be repositioned and the process repeated until a suitable pathway is identified.

At this point, the surgeon may remove the probe 12 from the stabilizer tube 24 (which is held in place by the articulating arm 424), and engage in direct visualization (e.g. look with his/her eyes) down the interior lumen 96 of the conduit 24 to ensure that the planned dilation pathway is clear of the genitofemoral nerve, for example (and anything else that would be problematic). In some embodiments, an optical source (e.g., light source, camera, etc.) may be advanced through the stabilizer tube 24 to aid in direct visualization of the planned dilation pathway.

Next, the dilator guide 26 may be fully inserted into the stabilizer tube 24 as described above. A dilator 28 is then advanced through the guide channel 116 corresponding to the selected pathway (e.g. guide channels 116, 116′, 116″). The dilator 28 then advances through the psoas muscle along the selected pathway. A surgical guide wire 30 may then be inserted through the dilator 28 into the target disc space (see e.g. FIG. 46).

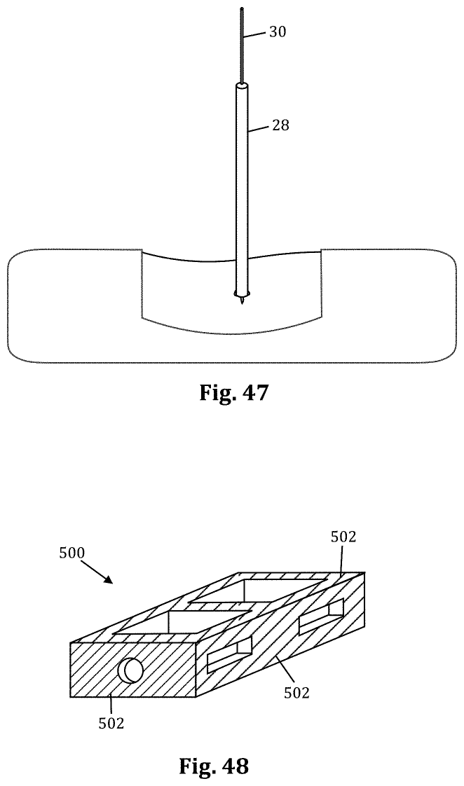

After the dilator 28 and K-wire 30 are placed, the dilator guide 26 may be removed from the stabilizer tube 24, leaving the stabilizer tube 24, dilator 28 and K-wire 30 in place. The stabilizer tube 24 may then be decoupled from the articulating arm 424 and removed from the incision, leaving the dilator 28 and K-wire 30 in place (see, e.g. FIG. 47). The lateral-access spine surgery may then continue by engaging in sequential dilation and retractor insertion, as is commonly known in the art of lateral access spine surgery.

Although the intraoperative ultrasound probe system 10 of the present disclosure is described herein as configured to facilitate navigation through tissue and neurovascular structure to determine an operative corridor to a surgical target site, in some embodiments the system 10 may be configured to locate and identify surgical implants (e.g., interbody implants, fixation plates, bone screws, rods, and the like), and differentiate between the surgical implants and anatomical structure. In such embodiments, the surgical implants may be modified or augmented to include one or more echogenic elements configured to reflect sound waves to make the surgical implants “visible” during ultrasound imaging. In some embodiments, the echogenic elements may comprise surface features including but not limited to (and by way of example only) notches, ridges, striations, and the like. In some embodiments, the surgical implants may be manufactured from echogenic material. By way of example only, FIG. 48 illustrates an exemplary interbody fusion implant 500 including a series of echogenic elements 502 (e.g. notches, ridges, striations etc.) configured to reflect sound waves to make the implant 500 “visible” during ultrasound imaging.

In some embodiments, the intraoperative ultrasound probe system 10 of the present disclosure may be configured to receive data collected through other modes, for example electromyography (EMG), integrate that data with ultrasound data, and display the combined data on the ultrasound image (e.g., as an additional overlay or an adjacent image) to create a confirmatory multi-modal display of the planned pathway and surrounding anatomical structures. By way of example only, a cannulated probe (e.g. cannulated probe 140 of FIGS. 19-22) may be provided in which the interior corridor 178 is electrically insulated to facilitate accurate delivery of electrical stimulation to a target site without shunting. In some embodiments, the surgical guide wire (e.g. K-wire 30) may also be electrically insulated to minimize shunting except for a portion of the tip that is exposed to accommodate directional electrical stimulation. By way of example, the interior corridor 178 may be sized and configured to accommodate passage of a blunt tip guide wire to avoid piercing tissue (e.g. nerve tissue) while being capable of puncturing the annulus of a target intervertebral disc (e.g. a thin and/or narrow blunt tip guide wire). Sequential dilation may then proceed using the placed guide wire. The guide wire may include a marker detectable by the ultrasound that enables visual tracking of the guide wire as it is advanced through tissue and may also indicate the direction in which the EMG stimulation is directed. EMG results collected during the advancement of the guide wire through the cannulated probe and tissue may then be displayed on the display unit 20 (e.g. as part of GUI screen 440 of FIG. 44 and/or GUI screen 442 of FIG. 45) to supply spatially coordinated EMG results into the same display as the ultrasound, thereby creating confirmatory multi-modal display. By way of example, the EMG results may be combined with the ultrasound image (e.g., as an additional overlay) or be displayed as a separate image adjacent to the ultrasound image.

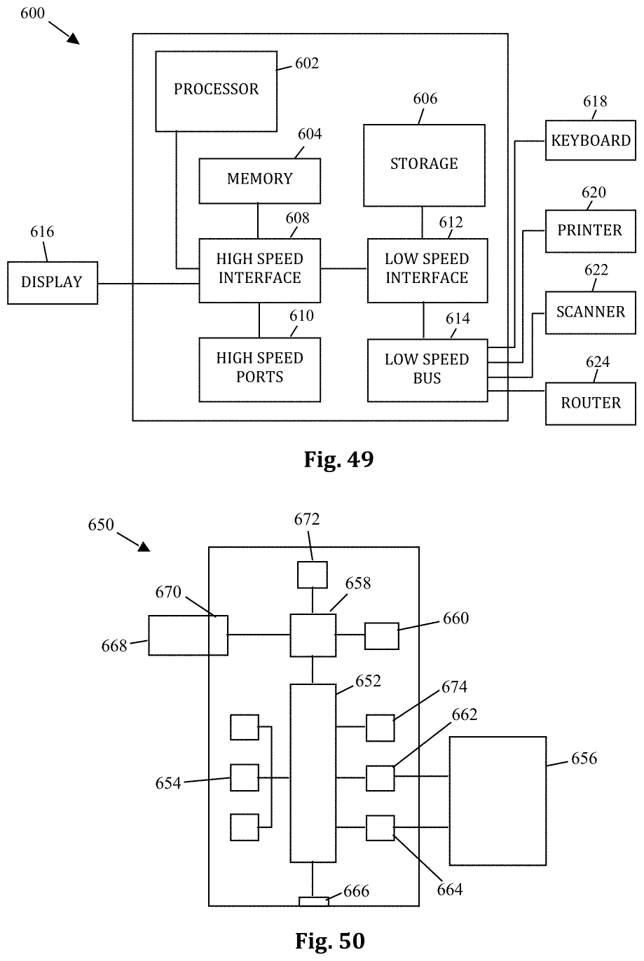

FIGS. 49-50 are example block diagrams of computer-implemented electronic devices 600, 650 that may be used to implement the systems and methods described in this document, as either a client or as a server or plurality of servers. Computing device 600 is intended to represent various forms of digital computers, such as laptops, desktops, workstations, personal digital assistants, servers, blade servers, mainframes, and other appropriate computers. Computing device 650 is intended to represent various forms of mobile devices, such as personal digital assistants, cellular telephones, smart-phones, and other similar computing devices. In this example, computing device 650 may represent a hand-held computing device 14, while computing device 600 may represent a physically larger system such as a stationary computer 14 and/or the mobile electronic device 300 of FIG. 37 and/or computing systems that serve as a cloud server. The components shown here, their connections and relationships, and their functions, are meant to be examples only, and are not meant to limit implementations described and/or claimed in this document.

Referring to FIG. 49, computing device 600 includes a processor 602, memory 604, a storage device 606, a high-speed interface 608 connecting to memory 604 and high-speed expansion ports 610, and a low speed interface 612 connecting to low speed bus 614 and storage device 606. Each of the components 602, 604, 606, 608, 610, and 612 are interconnected using various busses, and may be mounted on a common motherboard or in other manners as appropriate. The processor 602 can process instructions for execution within the computing device 600, including instructions stored in the memory 604 or on the storage device 606 to display graphical information for a graphic user interface (GUI) on an external input/output device, such as display 616 coupled to high-speed interface 608. In other implementations, multiple processors and/or multiple buses may be used, as appropriate, along with multiple memories and types of memory. For example, one or more graphics processing units (GPUs) may be used to accelerate the creation of images for display. Also, multiple computing devices 600 may be connected, with each device providing portions of the necessary operations (e.g., as a server bank, a group of blade servers, or a multi-processor system).

The memory 604 stores information within the computing device 600. By way of example only, the memory 604 may be a volatile memory unit, non-volatile memory unit, or another form of computer-readable medium, such as a magnetic or optical disk (for example).

The storage device 606 is capable of providing mass storage for the computing device 600. In one implementation, the storage device 606 may be or contain a non-transitory computer-readable medium (e.g., any computer-readable media except transitory, propagating signals), such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 604, the storage device 606, or memory on processor 602.

The high-speed interface 608 manages bandwidth-intensive operations for the computing device 600, while the low speed interface 612 manages lower bandwidth-intensive operations. Such allocation of functions is by way of example only. In one implementation, the high-speed interface 608 is coupled to memory 604, display 616 (e.g., through a graphics processor or accelerator), and to high-speed expansion ports 610, which may accept various expansion cards (not shown). In the implementation, low-speed interface 612 is coupled to storage device 606 and low-speed expansion port 614. The low-speed expansion port may include various communication ports (e.g., USB, Bluetooth, Ethernet, wireless Ethernet) and may be coupled to one or more input/output devices, such as a keyboard 618, a printer 620, a scanner 622, or a networking device such as a switch or router 624, e.g., through a network adapter.

The computing device 600 may be implemented in a number of different forms. For example, it may be implemented as a standard server, or multiple times in a group of such servers. It may also be implemented as part of a rack server system. In addition, it may be implemented in a personal computer such as a laptop computer. Alternatively, components from computing device 600 may be combined with other components in a mobile device, such as device 650 (FIG. 49). Each of such devices may contain one or more of computing device 600, 650, and an entire system may be made up of multiple computing devices 600, 650 communicating with each other.

Referring to FIG. 50, computing device 650 includes a processor 652, memory 654, an input/output device such as a display 656, a communication interface 658, and a transceiver 660, among other components. The device 650 may also be provided with a storage device, such as a microdrive or other device, to provide additional storage. The device 650 may further include one or more graphics processing units (GPUs) to accelerate the creation of images for display. Each of the components 650, 652, 654, 656, 658, and 660 are interconnected using various buses, and several of the components may be mounted on a common motherboard or in other manners as appropriate.

The processor 652 can execute instructions within the computing device 650, including instructions stored in the memory 654. The processor may be implemented as a chipset of chips that include separate and multiple analog and digital processors. Additionally, the processor may be implemented using any of a number of architectures. For example, the processor 652 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 650, such as control of user interfaces, applications run by device 650, and wireless communication by device 650.

The processor 652 may communicate with a user through control interface 662 and display interface 664 coupled to a display 656. The display 656 may be, for example, a TFT (Thin-Film-Transistor Liquid Crystal Display) display or an OLED (Organic Light Emitting Diode) display, or other appropriate display technology. The display interface 664 may comprise appropriate circuitry for driving the display 656 to present graphical and other information to a user. The control interface 662 may receive commands from a user and convert them for submission to the processor 652. In addition, an external interface 666 may be provided in communication with processor 652, so as to enable near area communication of device 650 with other devices. External interface 666 may provide, for example, for wired communication in some implementations, or for wireless communication in other implementations, and multiple interfaces may also be used.

The memory 654 stores information within the computing device 650. The memory 654 can be implemented as one or more of a non-transitory computer-readable medium or media (e.g. as described above), a volatile memory unit or units, or a non-volatile memory unit or units. Expansion memory 668 may also be provided and connected to device 650 through expansion interface 670, which may include, for example, a SIMM (Single In Line Memory Module) card interface. Such expansion memory 668 may provide extra storage space for device 650, or may also store applications or other information for device 650. Specifically, expansion memory 668 may include instructions to carry out or supplement the processes described above, and may include secure information also. Thus, for example, expansion memory 668 may be provided as a security module for device 650, and may be programmed with instructions that permit secure use of device 650. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, cause performance of one or more methods, such as those described above. The information carrier is a computer- or machine-readable medium, such as the memory 654, expansion memory 668, or memory on processor 652 that may be received, for example, over transceiver 660 or external interface 666.

Device 650 may communicate wirelessly through communication interface 658, which may include digital signal processing circuitry where necessary. Communication interface 658 may provide for communications under various modes or protocols, such as GSM voice calls, SMS, EMS, or MMS messaging, CDMA, TDMA, PDC, WCDMA, CDMA6000, or GPRS, among others. Such communication may occur, for example, through radio-frequency transceiver 660. In addition, short-range communication may occur, such as using a Bluetooth, WiFi, or other such transceiver (not shown). In addition, GPS (Global Positioning System) receiver module 672 may provide additional navigation- and location-related wireless data to device 650, which may be used as appropriate by applications running on device 650.

Device 650 may also communicate audibly using audio codec 674, which may receive spoken information from a user and convert it to usable digital information. Audio codec 674 may likewise generate audible sound for a user, such as through a speaker, e.g., in a handset of device 650. Such sound may include sound from voice telephone calls, may include recorded sound (e.g., voice messages, music files, etc.) and may also include sound generated by applications operating on device 650.

The computing device 650 may be implemented in a number of different forms, some of which are shown in the figure. For example, it may be implemented as a cellular telephone. It may also be implemented as part of a smart-phone, personal digital assistant, or other similar mobile device.

Additionally computing device 600 or 650 can include Universal Serial Bus (USB) flash drives. The USB flash drives may store operating systems and other applications. The USB flash drives can include input/output components, such as a wireless transmitter or USB connector that may be inserted into a USB port of another computing device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

While the inventive features described herein have been described in terms of a preferred embodiment for achieving the objectives, it will be appreciated by those skilled in the art that variations may be accomplished in view of these teachings without deviating from the spirit or scope of the disclosure. Although the various components have been described in terms of a system, is should be noted that the several components may be used independently of other components. Furthermore, although shown and described herein with respect to a specific example, it should be understood that the principles of the disclosure are not limited to the specific example described herein, and that various modifications and improvements may be made without departing from the score of the disclosure. For example, in some embodiments a retractor may be used to stabilize the probe 12. In some embodiments, direct visualization of the dilator/K-wire placement may be utilized prior to removal of the stabilizer tube. In some embodiments, the system may include integration of three-dimensional soft tissue mapping capabilities enabled by image-guided navigation. In some embodiments, robotic automation may be employed to enhance precision and efficiency. In some embodiments, several of the components described herein above may be used in a multimodal combination of ultrasound, robotics, and navigation to alleviate challenges of posterior minimally-invasive spine surgery.

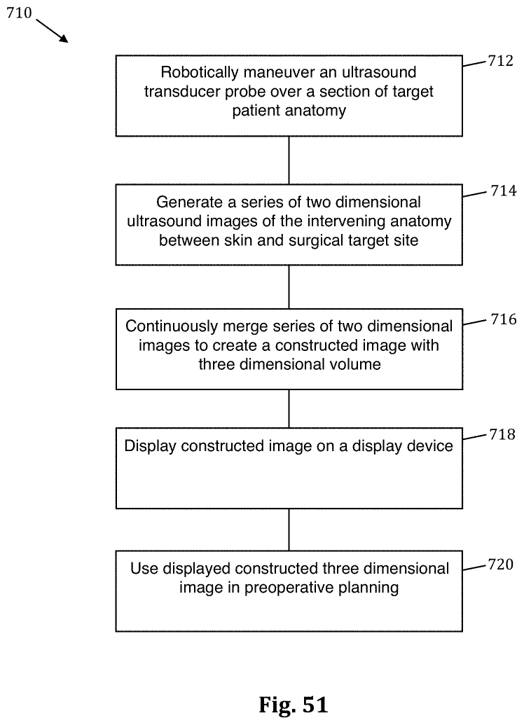

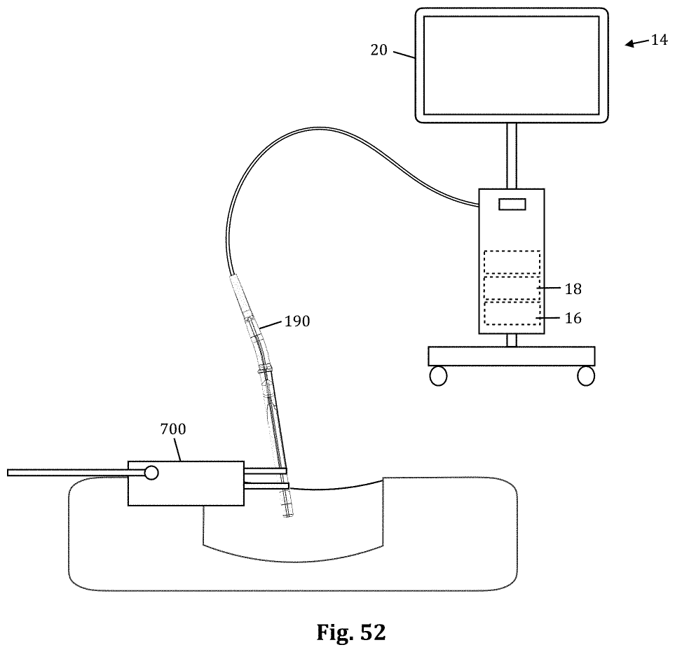

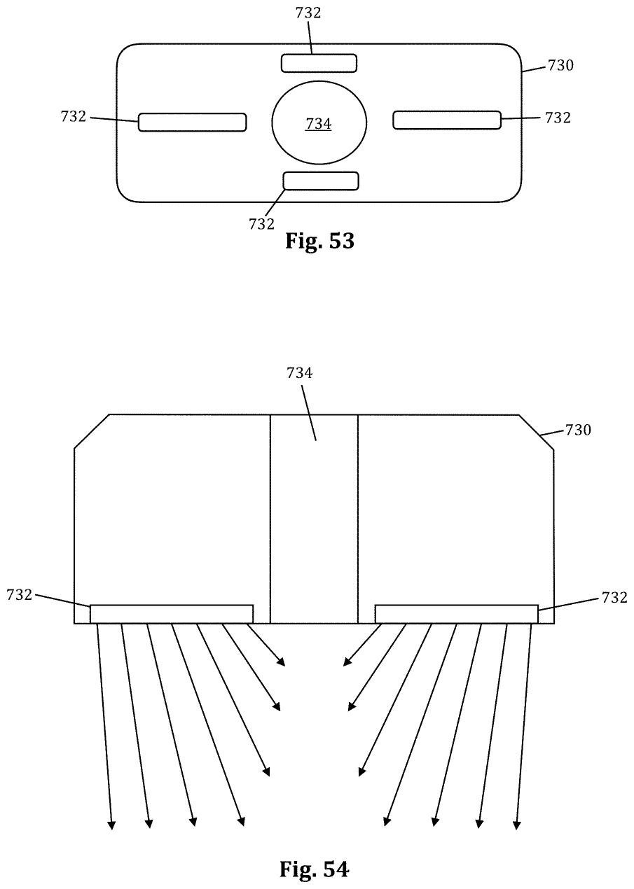

According to one example embodiment, the intraoperative ultrasound probe system 10 and/or one or more of the components described above (e.g. probe 190, software 18) may be paired with robotics to create a three-dimensional image of a patient's target anatomy with tissue differentiation without exposing the patient and practitioners to the X-ray radiation associated with certain commonly-used diagnostic imaging techniques (e.g. radiography, fluoroscopy, CT scans, etc.). By way of example, FIG. 51 illustrates a method 710 of generating a three-dimensional ultrasound image of intervening anatomy between a patient's dura and a surgical target site, which (for example) may be used in preoperative planning. By way of example, the first step 712 of the method 710 comprises directing a surgical robot 700 coupled with a probe 190 to perform an automated scan over a section of target patient anatomy (e.g., FIG. 52). According to a second step 714 of the method 710, the computer 16 may capture a series of two-dimensional ultrasound images (e.g. B-mode images) as the robot 700 maneuvers the probe 190 over a section of target anatomy in a controlled manner. According to a third step 716 in the method 710, the computer 16, by executing a set of software instructions (embodied in a non-transitory computer-readable medium and executable by the processor), may continuously merge the series of two-dimensional images to create a constructed image with three-dimensional volume. The computer 16 may also apply tissue differentiation functionality described above to provide visual identification to a user (e.g. surgeon) of specific anatomical structures (e.g. nerves) that may not be apparent from a standard B-mode ultrasound image. According to step 718 of the method 710, the constructed image may be displayed on a display unit 20. By way of example, the user may instruct the computer 16 to rotate the constructed three-dimensional image on the display unit 20. In some embodiments, this rotation may be facilitated by a touch-screen interface. In some embodiments, the position of the probe 190 may be tracked in space (e.g. by using at least one of optical, electromyography, and infrared tracking), for example by using a camera and array attached to the probe 190 and robotic unit 700.

By way of example, the three-dimensional image described above may be generated during a pre-operative patient visit to the hospital or surgical center, during the surgical procedure but prior to initial incision, or during the surgical procedure after the initial incision has been made. Once a three-dimensional image with tissue differentiation of the target anatomy has been constructed, the constructed image may be used in various pre-operative planning scenarios (e.g. step 720 of the method 710), which if executed may reduce the amount of time needed to complete the surgical procedure (regardless of when the planning occurs). For example, the constructed image may be used to pre-operatively determine a surgical trajectory from the patient's skin to the surgical target site (e.g. intervertebral disc space). The constructed image may also allow the surgeon to select anatomy for removal during the procedure to achieve decompression of and access to the target site. The tissue differentiation aspect of the constructed three-dimensional image enables the user to determine the location of certain areas to avoid (e.g. “no-fly zones”) while establishing an operative corridor to the surgical target site as well as determining which anatomy must remain intact. For example, the computer 16 may be instructed to automatically define the no-fly zone based on the presence of neural structures.

The computer 16 may be instructed to merge the constructed three-dimensional image with tissue differentiation with secondary images to provide more information to the user. For example, the computer 16 may be instructed to merge the constructed image with an intraoperative three-dimensional image collected from another source, a pre-operative CT scan, or a pre-operative MRI image. The computer 16 may match certain anatomical landmarks identified during the generation of the constructed image (e.g. during the continuous two-dimensional ultrasound scanning) with the corresponding anatomic landmarks on the secondary image to facilitate a merging of the images. The merged images are combined to create a comprehensive three-dimensional model of bone and soft tissue (for example) that may be intraoperatively reconciled.

In some embodiments, the intraoperative probe system 10 may include an ultrasound probe integrated or associated with a surgical instrument to generate ultrasound images while simultaneously placing the surgical instrument to achieve surgical objectives. The probe may be provided with a powerful transducer and placed on the skin above a target site comprising a posterior aspect or approach to the spine. The transducer may image deeper into the spine and with higher quality since the distance from the skin to the spine from a posterior approach is smaller than anterior or lateral, and also since there is less intervening anatomy between the skin and spine from a posterior approach. The probe with integrated or associated surgical instrument may be cannulated (for example as described above) to allow the surgical instrument to pass through the probe to achieve the surgical objectives, while also ensuring that the surgical instrument remains within the probe's “field of view” during use. Multiple probes employed at different angles to the target site may be used to gain a real-time awareness of the surgical instruments in space. The surgical instrument and/or ultrasound probe may be coupled to or integrated with a robotic element operated by the computer 16 for example to achieve controlled precision with one or more of motion, image capture, instrument positioning, and instrument operation to execute the procedure. Examples of instruments that may be used in such fashion include but are not limited to soft tissue dissection instruments, bone cutters (e.g. burrs), bone removal instruments, trocars, dilators, access cannulas, and/or K-wires or guide wires used to target and pierce the disc space.

By way of example only, the cannulated probe 730 referenced immediately above includes a plurality of ultrasound elements 732 surrounding a centrally located cannulation 734 extending through the probe 730 (e.g. FIG. 53). The size (e.g. diameter) of the cannulation may vary depending on the size of the instruments that need to pass through the cannulation as well as the surgical location and trajectory (e.g. anterior, posterior, lateral). The ultrasound elements 732 may be angled to create coverage across the cannulation area to enable real-time imaging even when an instrument is passed through the cannulation (e.g. FIG. 54).

In some embodiments, saline (or an alternative biologically compatible substance) may be used to fill a surgical incision to enable real-time ultrasound imaging during a surgical procedure. This is due to the fact that ultrasound imaging requires a continuous medium for the sound waves to travel through. During a surgical procedure, the patient's tissue may be disrupted, and as the patient tissue may be the continuous ultrasound medium, this medium may be interrupted which may in turn affect the ability to continuously generate an ultrasound image during the surgery. To enable continued imaging, saline (or an alternative biologically compatible substance) may be used to fill the surgical incision, enabling real-time ultrasound imaging to continue while instruments are being placed in the incision. During the procedure, there is a greater need for the ultrasound images to update continuously, and as such the need for a continuous medium is also great. The computer 16 may be instructed to determine (e.g. by way of sensors) when the medium is inadequate for continuous imaging and may be configured to either alert a user by an audio and/or visual warning element, or alternatively the computer 16 may be configured to automatically dispense an adequate amount of saline into the incision as is necessary to continue. To ensure the presence of enough saline, a device for saline storage and/or dispensing (e.g. comprising a bag of saline connected to a tub with a pump for saline delivery) may be provided and configured for automated use.

In some embodiments, adding image guidance to the ultrasound and navigated instruments of the system 10 enhances functionality by intraoperatively tracking the positioning of the ultrasound arrays in space and displaying that location on the constructed three-dimensional image with tissue differentiation. In order to achieve this, an optical, electromagnetic, or infrared camera may be strategically positioned to track the location in space of the ultrasound arrays at or near the distal end of the ultrasound probe in use. This location is then communicated to the computer 16 which applies this spatial awareness to the constructed image.

Once the three-dimensional image with tissue differentiation has been generated, a pre-operative plan has been determined, maintenance of adequate contact for imaging (e.g. continuous medium) has been confirmed, and certain instruments and/or components have been integrated into or associated with the robotic-enabled imaging apparatus, the computer 16 may use robotics to execute the pre-operative plan. For example, the computer 16 may be instructed to find specific incision locations and/or approach trajectories through the tissue as required by the pre-operative plan. The computer 16 may then instruct the robotic elements to manipulate attached or integrated instruments to execute the plan up to and including one or more steps in the surgical procedure. For example, the computer 16 may control the robot 700 to automatically navigate to the point of incision as determined by the plan, and then actually make an incision if the robot is also provided with a scalpel. Thus, surgical instrumentation, ultrasound imaging, navigation, and robotics are integrated into a singular user experience centered around a soft-tissue pre-operative plan made possible by the generation of the three-dimensional image with tissue differentiation of the patient's target anatomy.

In some embodiments, the intraoperative ultrasound probe system 10 may be configured to define avoidance areas or “no-fly zones” intraoperatively via real-time identification of nerves and soft tissue using the differentiation feature of the software 18. For example, a burr may be used to cut bone to decompress the disc in an incision full of saline to enable real-time ultrasound imaging as described above. During the advancement of the burr toward the target site, the computer 16 is executing the ultrasound imaging and tissue differentiation functionalities and can thus determine the location of the burr in space relative to patient anatomy. If the computer 16 determines that tissues of concern (e.g. nerves) are being approached by the burr, further advancement of the burr may be disabled immediately and/or other movement into that area may be restricted. In this example, robotic haptics may be combined with an ultrasound probe and surgical instruments (e.g. burr). Image guided navigation may also be used as a multimodal confirmation of the burr's positioning in space.

In some embodiments, the three-dimensional image construction techniques may be used in surgical approaches other than the posterior one referenced by way of example only above. For example, the image construction technique may be used in relation to a lateral approach as well. In such a case, the three-dimensional constructed image may be of the psoas muscle (for example) so that the user may select a working corridor in a three-dimensional space. The robotics and navigation components may then be utilized to facilitate access to the surgical target site in much the same way as described above.

In addition to the examples described above, the three-dimensional image construction technique may be used to expedite, simplify, and automate the initial image registration process by using navigation and ultrasound to identify and align points.

Additionally, the three-dimensional image construction technique described above may be used for dynamic referencing without the need for a fiducial placement. In this instance, the three-dimensional image and spatial tracking function may be used to automatically track and register the location of an instrument in space. By registering the three-dimensional constructed image to identified patient anatomy, the computer 16 may be able to sense or recognize if/when a patient has moved and automatically shift the constructed image (and any merged images) to reflect that patient movement.

What is claimed is:

1. A method of generating a three-dimensional ultrasound image of intervening anatomy between a patient's dura and a surgical target site, comprising:
maneuvering a distal end of at least one ultrasound probe along the outside of the patient's dura over a section of target patient anatomy, the ultrasound probe having a proximal end, a distal end, an electronic communication element, and a transducer array positioned near the distal end, the transducer array including at least one emitting element configured to emit high-frequency sound waves within a proximity of the distal end and in a direction away from the distal end, the transducer array further comprising at least one sensing element configured to receive reflected sound waves and convert the reflected sound waves to radio frequency data;
performing ultrasound imaging to generate a series of two-dimensional B-mode images of the intervening anatomical structures from the radio frequency data obtained by the probe;
continuously merging the series of two-dimensional images to create a constructed image with three-dimensional volume; and
displaying the constructed image of the intervening anatomical structure on a display device;
wherein the constructed image includes a highlighted position of at least one of nerve, muscle, and bone.

2. The method of claim 1, wherein the ultrasound probe is associated with a robotic element.

3. The method of claim 2, wherein the ultrasound probe is robotically maneuvered over the section of target patient anatomy in a controlled manner.

4. The method of claim 1, wherein the constructed image is rotatable on the display unit.

5. The method of claim 1, wherein the display unit comprises a touch-screen interface.

6. The method of claim 1, further comprising the step of:
tracking the spatial position of the at least one ultrasound probe during generation of the constructed image.

7. The method of claim 6, wherein the spatial position of the at least one ultrasound probe is tracked using at least one of optical tracking, electromyography, and infrared tracking.

8. The method of claim 1, wherein the constructed image is generated at least one of before and during a surgical procedure.

9. The method of claim 1, further comprising the step of
merging the constructed image with at least one of a CT scan image and a MRI image to create a comprehensive three-dimensional model of the target patient anatomy.

10. The method of claim 9, wherein the comprehensive three-dimensional model includes bone and soft tissue.

11. The method of claim 9, wherein the constructed image and at least one of a CT scan and a MRI image are merged using anatomical landmarks.

12. The method of claim 1, wherein the at least one ultrasound probe is associated with a surgical instrument.

13. The method of claim 1, further comprising the step of:
analyzing the constructed image to create a pre-operative plan including at least one of incision location and approach trajectory to the surgical target site.

14. The method of claim 13, wherein the pre-operative plan is executed by robotic elements.

15. The method of claim 1, further comprising the step of:
analyzing the constructed image to identify avoidance areas around at least one of the incision location and approach trajectory to the surgical target site.
